(19) United States
(12) Patent Application Publication
(10) Pub. No.: US 2026/0162786 A1
(43) Pub. Date: Jun. 11, 2026

(54) EMULATING CLINICAL TRIAL DATASETS FROM PUBLISHED ANALYSIS STATISTICS

(71) Applicant: Telperian, Inc., Austin, TX (US)

(72) Inventor: Brian Paul Hobbs, Austin, TX (US)

(21) Appl. No.: 19/410,946

(22) Filed: Dec. 5, 2025

Related U.S. Application Data
(60) Provisional application No. 63/728,488, filed on Dec. 5, 2024.

(51) Int. Cl. G16H 10/60; G06N 20/20; G16H 10/20

(52) U.S. Cl. CPC G16H 10/60; G06N 20/20; G16H 10/20

(57) ABSTRACT

Provided is a process, including: obtaining a publication reporting statistics resulting from a clinical trial; generating, without having access to real patient records in the clinical trial, a simulated clinical trial dataset comprising simulated patient records consistent with the statistics in the publication by determining constraints on the simulated patient records from the publication and then, iteratively, until a stopping condition is detected, generating a new set of candidate simulated patient records by adjusting an initial set or current set of candidate simulated patient records, scoring the new set of candidate simulated patient records based on satisfaction of the constraints, and selecting a subset of the new set of candidate simulated patient records based on the scores as the current set of candidate simulated patient records; and storing at least some of the current set of candidate simulated patient records as the simulated patient records.

### CROSS-REFERENCE TO RELATED APPLICATIONS

This patent claims the benefit of U.S. Provisional Patent Application 63/728,488, filed Dec. 5, 2024, titled EMULATING CLINICAL TRIAL DATASETS FROM PUBLISHED ANALYSIS STATISTICS. The entire content of each afore-listed earlier-filed application is hereby incorporated by reference for all purposes.

### BACKGROUND

### 1. Field

The present disclosure relates generally to artificial intelligence used in the generation and analysis of synthetic data related to clinical studies.

### 2. Description of the Related Art

Clinical trial data is useful for assessing the safety and efficacy of therapeutic interventions. But access to detailed, patient-level data in published studies is often restricted due to legal, ethical, and proprietary constraints. This lack of access is particularly acute in cases where competing organizations, such as pharmaceutical companies, cannot share data, or where privacy regulations limit data dissemination. Even when summary statistics are published, they are typically insufficient for further analysis, as they fail to capture nuanced patient-level variability, multivariate relationships, and critical time-dependent outcomes. These gaps can create significant challenges for researchers and stakeholders who need robust datasets to make informed decisions.

The inability to access or analyze detailed clinical data can impose substantial costs. For trial designers, the absence of relevant benchmarks and comprehensive data often results in inefficient studies, increased expenses, and unnecessary duplication of effort. Regulatory agencies and sponsors may face delays or uncertainties in evaluating therapeutic efficacy and safety. Furthermore, decisions based on incomplete datasets risk overlooking crucial insights, such as subgroup-specific responses or adverse event patterns, potentially leading to missed opportunities to improve patient outcomes and optimize treatment protocols. The absence of accessible, detailed clinical data ultimately hinders innovation, slows drug development, and increases the financial burden on the biomedical industry.

Software tools for conducting meta-analyses of multiple clinical studies are available. The tools, however, generally use only a fraction of the information reported from a clinical trial, typically only point estimate and 95% confidence interval of the primary endpoint. Statistical tools for meta-analysis often assess the heterogeneity of these univariate statistical summaries. The current state is generally intrinsically limited by the absence of multivariate assessment which would facilitate the much more comprehensive understanding of the study. This includes, but is not limited to (which is not to imply other lists are limiting) evaluating differences in patient enrollment, analysis of relationships between variables and outcomes that are not directly reported, results of subpopulations that are not explicitly reported, and differences in the impact of potentially important predictive and prognostic determinants.

Moreover, with current software tools investigators are often precluded from comparing distributions of patient characteristics and their associations with outcomes across studies (which is not to imply embodiments are limited to systems that mitigate this issue or other issues described herein). In addition, for time-to-failure endpoints (such as overall survival, time-to-progression, time-to-recurrence, progression-free survival, disease-free survival duration of response, and relapse-free survival) many forms of statistical meta-analysis software for univariate summary statistics preclude evaluations of outcome distributions over time with modifications to the enrollment rate and duration of follow-up.

Lacking tools devised to integrate the actual statistical evidence and extract intelligence for understanding the landscape, current benchmarks for toxicity and response in early phase trials are commonly defined by expert opinion. Because the participating clinicians cannot be expected to have read and fully understood every statistical analysis report from every relevant trial, current study designs commonly fail to account for results of recent trials. Furthermore, relying on published trial reports to guide decision-making is effectively impossible as the trial summary statistics do not account for patient heterogeneity nor differences in patient enrollment and follow-up.

The lack of technology for evidence synthesis and strategic intelligence limits the capacity of trialists and stakeholders to learn from previous studies. This continues to drain the biomedical industry for which costs increase over time while success rates decline. Regulatory approval for oncology drugs (<4%), for example, remains among the lowest in biomedicine despite considerable investment in recent decades. It is helpful to the future credibility of the clinical trials industry and its various stakeholders to evolve from the current state which relies on expert speculation informed by univariate reviews of statistical summaries due to constraints imposed by existing tooling. None of the proceedings should be read to suggest that these univariate reviews of statistical summaries or anything else is disclaimed or disavowed, as the present techniques may be used in conjunction with those approaches.

### SUMMARY

The following is a non-exhaustive listing of some aspects of the present techniques. These and other aspects are described in the following disclosure.

Some aspects include a process with steps including: obtaining a publication reporting statistics resulting from a clinical trial; generating, without having access to real patient records in the clinical trial, simulated patient records consistent with the statistics in the publication by determining constraints on the simulated patient records from the publication and then, iteratively, until a stopping condition is detected, generating a new set of candidate simulated patient records by adjusting an initial set or current set of candidate simulated patient records, scoring the new set of candidate simulated patient records based on satisfaction of the constraints, and selecting a subset of the new set of candidate simulated patient records based on the scores as the current set of candidate simulated patient records; and storing at least some of the current set of candidate simulated patient records as the simulated patient records consistent with the statistics in the publication.

Some aspects include a tangible, non-transitory, machine-readable medium storing instructions that when executed by a data processing apparatus cause the data processing apparatus to perform operations including the above-mentioned process.

Some aspects include a system, including: one or more processors; and memory storing instructions that when executed by the processors cause the processors to effectuate operations of the above-mentioned process.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1 is a flow chart of a process for defining clinical trials, related trial arms, and benchmarks, including the ingestion of published analyses and calibration to reported statistics, in accordance with some embodiments.

FIG. 2 is a flow chart of a simulation algorithm for iteratively generating, evaluating, and refining synthetic datasets based on statistical benchmarks, in accordance with some embodiments.

FIG. 3A is a flow chart of the proposal step for generating candidate trial arm datasets, including imputing missing values and aligning synthetic patient profiles to study endpoints, in accordance with some embodiments.

FIG. 3B is a flow chart of the incrementation step for generating synthetic datasets by probabilistically adjusting variables and creating partially observed datasets, in accordance with some embodiments.

FIG. 4 is a flow chart of a process for statistical inference and meta-analysis, involving merging synthetic datasets, applying criteria, and analyzing empirical distributions, in accordance with some embodiments.

FIG. 5 is a flow chart of a process for merging top-ranked synthetic datasets across trials by harmonizing variables, iteratively combining datasets, and imputing missing values, in accordance with some embodiments.

FIG. 6 is a flow chart of a process for unsupervised graphical ensemble machine learning, including creating embeddings, clustering patients, and profiling clusters, in accordance with some embodiments.

FIG. 7 is a flow chart of a supervised ensemble machine learning process that uses cross-validation to rank models by concordance and generate predictions for patient profiles, in accordance with some embodiments.

FIG. 8 is a flow chart of a semi-supervised ensemble machine learning process for predicting outcomes, building graphical models, and validating patient profiles across datasets, in accordance with some embodiments.

FIG. 9A is a flow chart of the creation of synthetic datasets, prediction models, and virtual clinical trial designs with defined parameters and endpoints, in accordance with some embodiments.

FIG. 9B is a flow chart of a replicate algorithm for virtual trial simulation, including generating virtual cohorts, predicting outcomes, and summarizing probabilities of success, in accordance with some embodiments.

FIG. 10 is a block diagram of an example system for generating synthetic patient-level data from a clinical study publication, in accordance with some embodiments.

FIG. 11 is a flow diagram of an example method for generating a synthetic dataset from a clinical study publication, in accordance with some embodiments.

FIG. 12 is a flow diagram of an example method for generating a synthetic dataset from a clinical study publication, in accordance with some embodiments.

FIG. 13 illustrates a system diagram of an exemplary computing device that may be configured to execute the method, in accordance with some embodiments, in accordance with some embodiments.

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION OF CERTAIN EMBODIMENTS

To mitigate the problems described herein, the inventors had to both invent solutions and, in some cases just as importantly, recognize problems overlooked (or not yet foreseen) by others in the field of synthetic data generation. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

It has been recognized that advances in computer algorithms, simulations techniques, and computational infrastructure can facilitate the emergence of powerful analysis and simulation tools. Some embodiments leverage, combine, and extend upon, these advances in computational capacity to emulate clinical trial datasets from collections of statistical analysis results as well as facilitate machine learning of the resultant database to extract strategic intelligence for numerous types of stakeholders. As noted above, often the underlying patient level data of published clinical trial results are not made available. Some embodiments emulate that underlying patient level data to produce synthetic data that can be analyzed to produce additional insights, like those that could be extracted from the original unpublished patient level data. In some embodiments, this emulation is done based upon the limited statistics and other information in published studies without having access to the original patient level data, for instance, based upon population statistics published in those studies.

Statistical analysis summaries that result from reports of data collected in clinical trials constitute a set of constraints that inform plausible distributions of the underlying, un-available patient-level data. These constraints, along with information about when patients were evaluated during the course of follow-up and the duration required to achieve final accrual, can be used to evaluate candidate arrangements of the underlying patient-level data. Benchmarks may be established based on actual observed patient-level datasets, structured tables of statistical summary statistics reported in publications, or other methods that would support the implementation of analysis on both candidate synthetic datasets and benchmark external data. Patient-level data that is external to a trial but relevant to the study may be uploaded to create a benchmark. Benchmarks may also be created by uploading tables of statistical summary statistics reported in publications. This method may be advantageous because it does not require patient-level data to generate a synthetic dataset. For example, if an article reports a subgroup that compares overall survival for patients older than 65 between Drug A and control arms with a resultant hazard ratio for the actual data analysis being 0.81 with a 95% confidence interval for a range of 0.77 to 0.85. The system implements the same analysis upon evaluating a candidate synthetic dataset that may estimate the hazard ratio to be 0.83 with a 95% confidence interval for a range of 0.79 to 0.87. Any differences between the actual and synthetic results may represent bias in the candidate synthetic data. Bias between the actual and synthetic result may be minimized by optimizing the simulation algorithm among all benchmark analyses. Regardless of the method of creation for a benchmark, analyses may be performed on both candidate and benchmark external data and any resulting bias assessed.

Some embodiments leveraging structural knowledge of clinical trial design and statistical analysis techniques and standards for reporting clinical trial results combined to provide computer software (e.g., in a package include executable code, libraries, and documentation) to reconstitute datasets that are likely to have been observed in a clinical trial without access to the patient-level data. The datasets are reconstituted or otherwise emulated (or more generally, simulated) on the basis of characteristics of the trial design (e.g. phase of development, statistical design, enrollment duration, latency period between patient visits, planned sample size and number of events, measured endpoints) and collections of statistical summaries described in various statistics in trial reports (results, figures, tables, appendices). The result does not necessarily reveal the actual patient level data in the clinical trial, but it does support analyses that are expected to yield results like those that would be obtained if that unavailable data was further analyzed. The reconstituted clinical trial datasets are referred to as “synthetic” datasets of the clinical trial. Of note, unlike some other forms of synthetic data, this synthetic data is generated without access to the unpublished patient-level data of the clinical trial, e.g., showing for each patient, a unique identifier, interventions applied, measurements taken, and outcomes. Each candidate synthetic dataset is scored according to its overall “concordance” with the breadth of statistics contained in the analysis report. Computational algorithms may be used for simulation facilitate the rapid generation and scoring of millions of candidate synthetic datasets, which may be ranked according to statistical measures of bias and concordance with reported statistics. Top scoring datasets may then be saved and integrated for further analysis, in some embodiments.

Translating trial analysis summary statistics into plausible (e.g., in the sense that analysis of a population of the same produces results like those that would be obtained from analyzing the unpublished data) individual patient-level datasets may unlock the potential for numerous potential applications of statistical modeling, machine learning, and trial simulation that require patient-level data and thus not accessible to conventional meta-analytic techniques. Moreover, by simulating a collection of possible datasets and extracting intelligence through analyses of highly concordant subsets, the method circumvents numerous legal and ethical barriers that block access to the actual underlying patient-level data itself, which is only available to restricted few stakeholders. These stakeholders are mainly federal regulatory bodies who do not share data as a policy with stakeholders.

The software may additionally integrate the top ranked reconstituted clinical trial datasets across multiple clinical trials into an integrated database that can be used for interrogating multiple trials, therapies, disease indications, and drug classes. The software may include machine learning tools devised to extract intelligence from the resultant integrated evidence to describe distributions of outcomes among trials, endpoints, therapies, and subgroups as well as define probabilistic distributions of relationships between variables not directly described in the trial analysis reports. Virtual trial simulation algorithms enable counterfactual evaluations and comparisons of candidate future trial designs based on the integrated evidence as well as benchmarks for underperformance and outperformance in sequential analyses. Moreover, numerous clinical trial design decisions and assumptions (number of sites, activation schedule, sample size, interim analysis schedule and final analysis date, number of arms, eligibility criteria, statistical analysis methods, etc.) may be refined and calibrated for optimal performance by counterfactual virtual trial simulation devised to measure the probability of success for any design choices from evidence contained within synthetic datasets.

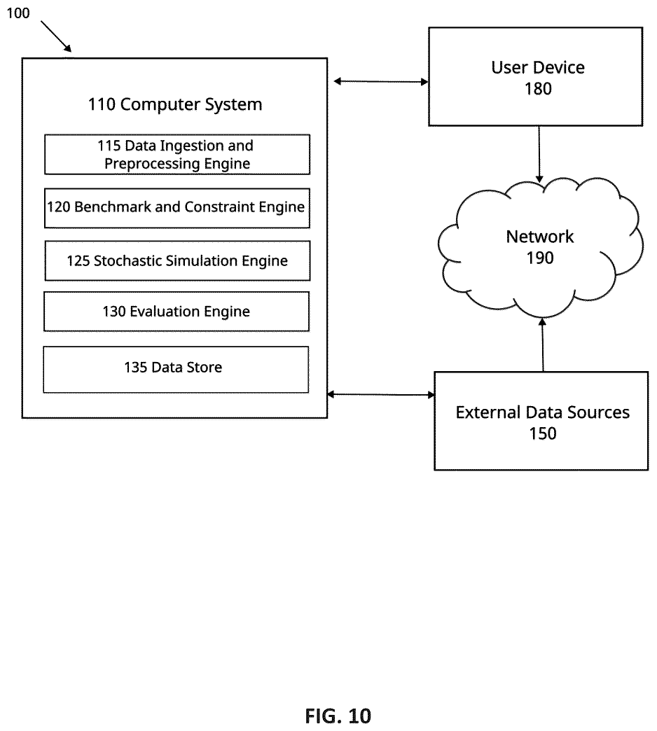

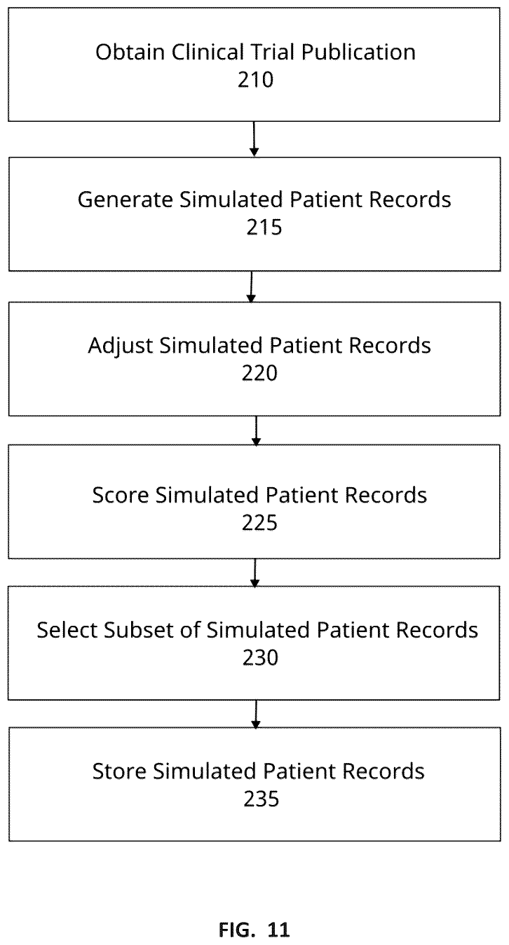

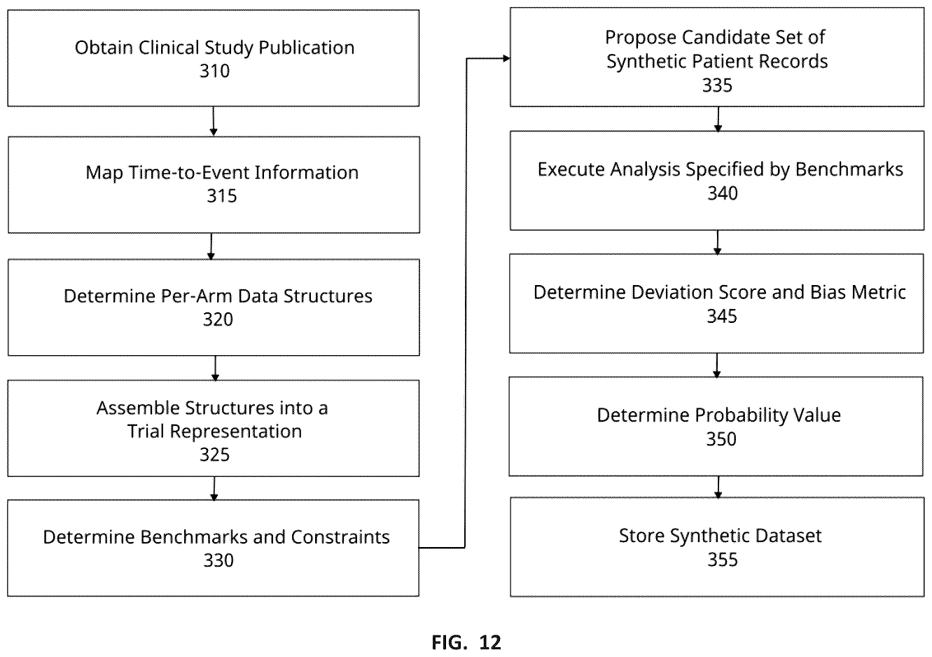

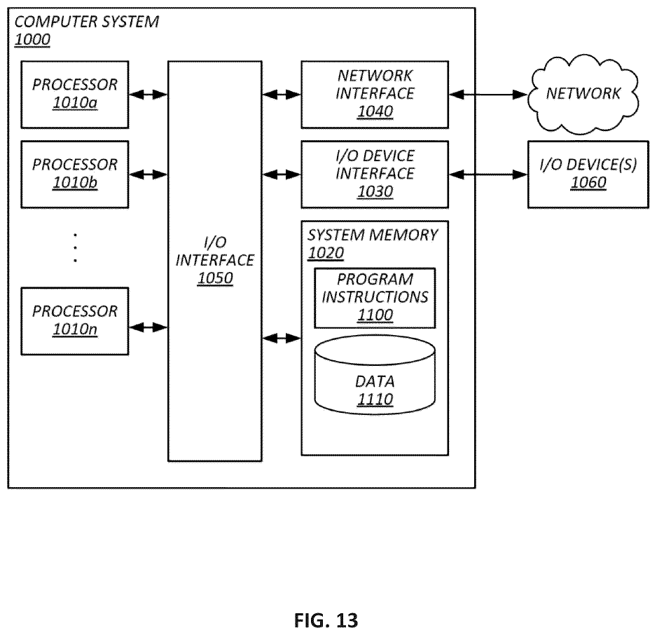

The processes and code in the figures that follow may be executed on one or more computing devices like that shown in FIG. 13, e.g., in systems like those described with reference to FIG. 10 below, running processes like those described with reference to FIGS. 11-12 below. For example, some embodiments may operate with a client server architecture in which a server system with one or more of those computing devices cooperates, for example in cloud infrastructure, to perform the processes described herein in response to inputs from a client computing device accessing those tools remotely over a network like the internet and showing results on that client computing device, for example on a user interface.

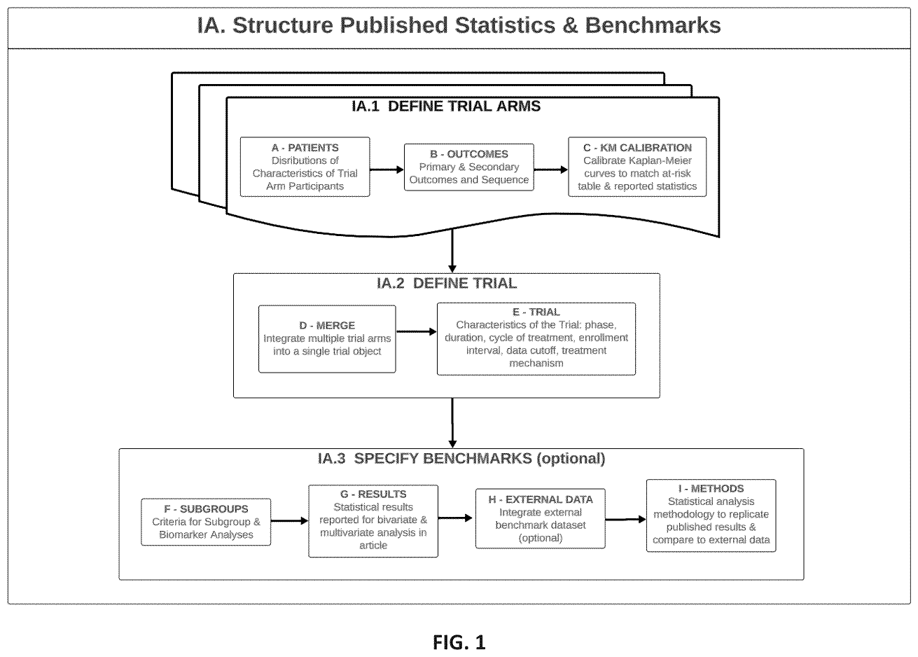

FIG. 1 is a flow chart of an example process for defining a clinical trial, its related trial arms, and specifying benchmarks following trial definition. When defining trial arms, distributions of patient characteristics, such as age, gender, biomarker status, and comorbidities, may be ingested along with primary and secondary outcome data. The outcomes may include metrics such as survival rates, progression-free intervals, and response rates. These outcomes may be tied to the trial arm participants using metadata that associates patient-level characteristics with endpoint results. In some embodiments, the process of FIG. 1 may be executed by a human being reading a PDF of a published clinical trial study and inputting the various fields into the processes that follow. In some embodiments, this may include encoding those values in a data structure like those described below. In some embodiments, the PDF of the clinical trial publication may be processed with optical character recognition algorithms, large language models, and the like to extract the various fields described. Tools like WebPlotDigitizer or custom graphical tools for extracting the KM curves from plots and generating the closets possible individual-patient level dataset may be used to extract coordinates of curves in published graphs, for example. In some cases, the output of the process of FIG. 1 may supply the constraints by which plausible emulated data is generated in the figures that follow. In some embodiments, the analyses and generation processes described below may be dynamically scaled based upon the amount of available computing resources. For example, some embodiments may adjust the level of search for plausible concordant data sets based on the number of available cores to perform the described analysis in search. Some embodiments may have between 128 and 512 cores inclusive, for example, to concurrently perform the processes described below. Some embodiments may have more cores and some embodiments may have less. In some cases, these may be CPU cores or some embodiments may use heterogeneous computer architectures, for instance, including graphics processing units, tensor processing units, AI accelerator chips, and the like.

Subsequent to ingesting this data, calibration steps may be performed. Kaplan-Meier curves may be used to align the data with at-risk tables, reported statistics, and other analyses. Once the trial arms are defined, they may be integrated into a single trial object. This trial object may contain parameters including the trial phase (e.g., Phase I, II, or III), duration, cycle of treatment, enrollment intervals, data cutoff points, and the treatment mechanism of action (e.g., targeted therapy, chemotherapy).

Benchmarks may optionally (which is not to imply other features are required) be specified at this stage (the reference to steps being “optional” should not be read to imply that other steps or features are required in all cases). These benchmarks may include criteria for subgroup analyses (e.g., age-specific or biomarker-defined populations), bivariate or multivariate statistical results, external benchmark datasets (e.g., real-world data sources), and methodologies for replicating published results. The benchmarks may further facilitate comparisons between synthetic and external data for validation and exploratory analyses.

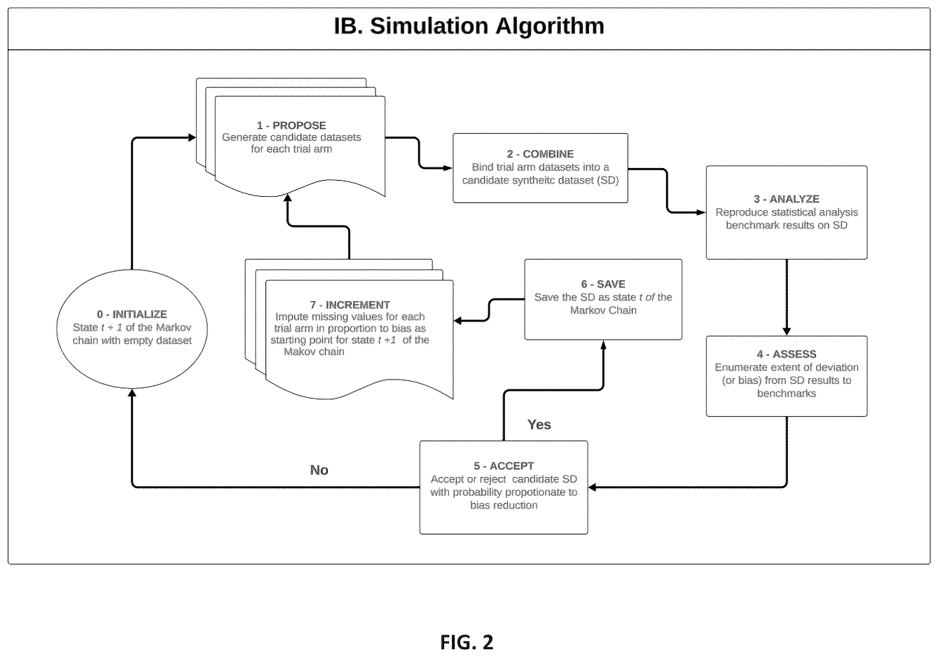

FIG. 2 is a diagram describing an example simulation algorithm used to generate synthetic datasets, e.g., constrained by the data produced by the process of FIG. 1, which may be taken as an input. The process, in some embodiments, begins with initializing the algorithm at state t+1 of a Markov chain, starting with an empty dataset. Candidate datasets may be generated for each trial arm using probabilistic models or other computational methods. These candidate datasets may include imputed patient characteristics and outcomes constrained by the reported statistical summaries of the trial.

Once candidate datasets for all trial arms are generated, in some embodiments, the trial arm datasets may be combined to form a synthetic dataset representing the entire trial. This synthetic dataset may then undergo an analysis step to reproduce statistical benchmark results. The synthetic datasets are evaluated for deviation or bias from the benchmark statistics. For example, the algorithm may assess whether key metrics deviate beyond acceptable thresholds from the reported results.

Based on this evaluation, in some embodiments, the algorithm may probabilistically accept or reject the synthetic dataset. If rejected, in some embodiments, the simulation restarts at t+1 with an empty dataset. If accepted, the synthetic dataset is saved as state t of the Markov chain. Missing values in the dataset may then be imputed, using statistical methods to ensure consistency with benchmark constraints. The process, in some embodiments, iterates, generating successive synthetic datasets and refining their concordance with reported results until a desired level of accuracy and bias reduction is achieved.

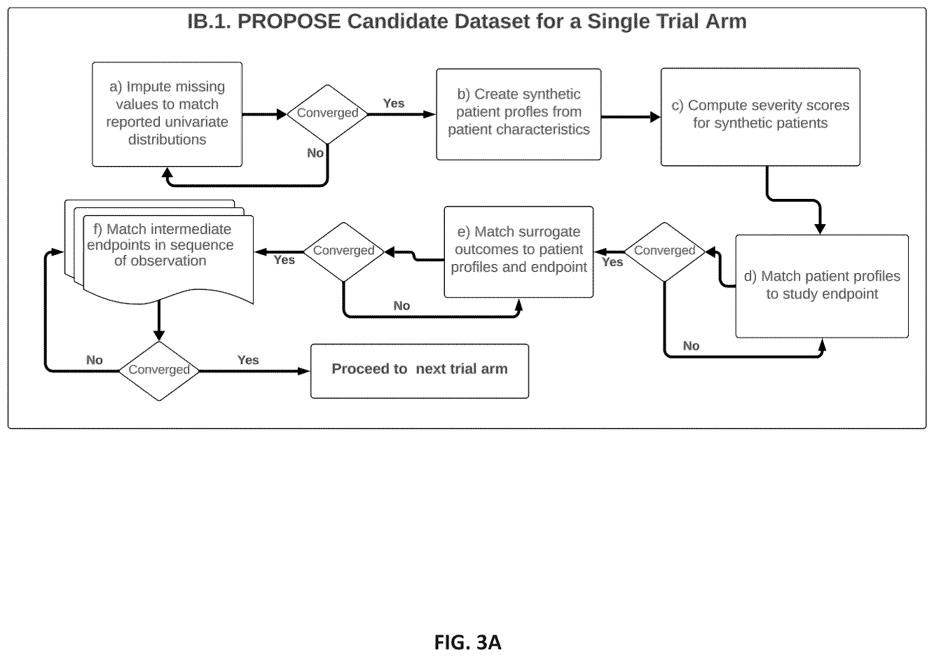

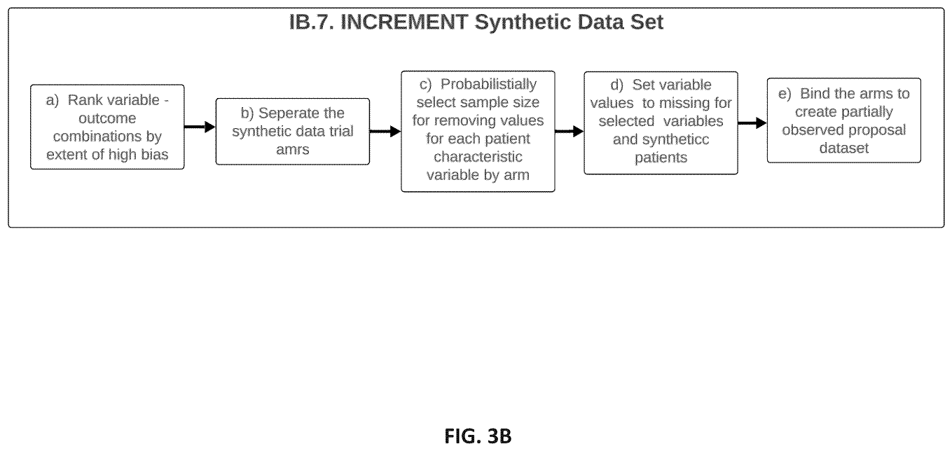

As illustrated, the higher-level descriptions of various steps in FIG. 2 are elaborated upon in subsequent figures. For example, step 1 in FIG. 1 is expanded upon in the process shown in FIG. 3A. A similar pattern follows in FIG. 3B, which expands on step 7 in the process of FIG. 2.

FIG. 3A depicts a detailed diagram of an embodiment of the proposal step for generating a candidate dataset for a single trial arm. Initially, in some embodiments, missing values in the trial arm data are imputed to match reported univariate distributions, such as distributions of patient age, biomarker positivity, or treatment adherence rates. If these distributions do not converge to match the statistical constraints, in some embodiments, the proposal step is restarted.

Once univariate distributions converge, the algorithm, in some embodiments, generates synthetic patient profiles. Each profile includes multiple characteristics, such as demographic data, baseline biomarker levels, and treatment assignment. Severity scores for individual synthetic patients may then be computed based on the characteristics and matched to study endpoint results (e.g., progression-free survival or response rates).

If patient profiles fail to align with endpoint results, in some embodiments, the algorithm iterates until convergence is achieved. Surrogate outcomes, such as intermediate progression markers or adverse event metrics, may also be matched to synthetic patient profiles. Once the trial arm data successfully converges, the process may proceed to the next trial arm.

In some cases, the described process may generate a set of emulated data (a type of synthetic data created based on statistics describing underlying data but without full access to that underlying data) that plausibly could have resulted in the published analysis in a paper describing a clinical trial, while accounting for constraints from the physical world, like that patients cannot progress after they die, or males tend to be more likely to have heart disease as they age, or the like. These constraints (both those published and those from the physical world) may drive the search for emulated data sets of emulated patient level data. This is challenging for a variety of reasons. In some cases, an objective function accounting for these constraints is non-differentiable, meaning that techniques commonly used in machine learning, like gradient descent, cannot be used in some cases. Further the process is informed of the results of the trial arms, but is not explicitly informed of how to match across variables in emulated patient level data. Some embodiments may execute more than 10,000, like more than one million iterations of the described loops to arrive a set of emulated data.

FIG. 3B provides a detailed depiction of an embodiment of the incrementation step for synthetic data generation. During this step, in some embodiments, the algorithm may rank variable-outcome combinations by the extent of observed bias or deviation from benchmark statistics. For example, variables with the greatest deviation may be prioritized for correction.

The algorithm may probabilistically select a sample size for imputation, removing or holding certain variable values as “missing” to simulate partially observed data. Selected variables and synthetic patients may then be adjusted to reflect realistic distributions, constrained by reported trial statistics. The adjusted trial arms may then be re-bound to create an updated synthetic dataset. This process, in some embodiments, iterates until the synthetic dataset converges with reported benchmarks across all trial arms.

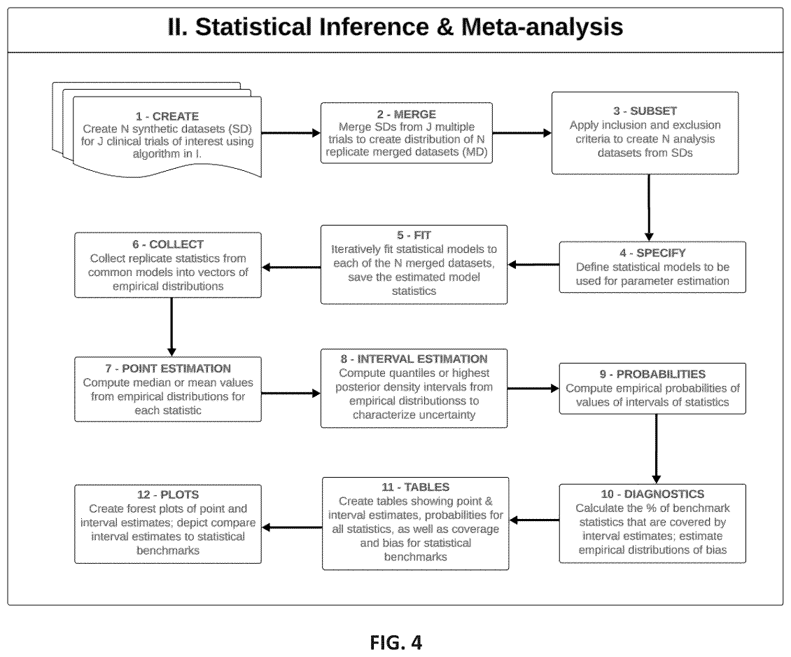

FIG. 4 depicts a flow chart of an example process of statistical inference and meta-analysis. Initially, in some embodiments, an algorithm is employed to create a plurality of synthetic datasets for clinical trials of interest. These synthetic datasets, in some embodiments, may represent reconstructed patient-level data generated from statistical summaries and trial metadata.

The synthetic datasets from multiple trials, in some embodiments, may then be merged into a distribution of replicated merged datasets. This merging process, in some embodiments, accounts for variations in trial characteristics, including patient demographics, treatment regimens, and reported outcomes, ensuring harmonization for cross-trial comparisons. Once the datasets are merged, inclusion and exclusion criteria may be applied to define analysis-ready datasets. For example, inclusion criteria may target specific subgroups of interest, such as stratified biomarker-defined populations, while exclusion criteria may remove synthetic patients with inconsistencies or irrelevant attributes.

The process, in some embodiments, allows users to specify statistical models for parameter estimation. These models may include generalized linear models, multivariate regression, or machine learning-based methods tailored to the objectives of the analysis. Each model, in some embodiments, is iteratively fit to the replicated merged datasets, and the estimated parameters for each iteration are saved. These replicated parameters, in some embodiments, are compiled into empirical distributions, enabling comprehensive statistical assessments.

Empirical distributions, in some embodiments, are analyzed to compute descriptive statistics, including mean, median, variance, and other summary measures. Additional analyses may include interval estimation, wherein quantiles (e.g., 5th and 95th percentiles) or highest posterior density intervals are computed to characterize uncertainty. The method, in some embodiments, may also compute empirical probabilities associated with specific parameter values or statistical intervals, allowing users to assess the likelihood of outcomes.

Diagnostics, in some embodiments, may then be performed on the computed statistics to validate their accuracy and reliability. These diagnostics may include calculations of benchmark coverage, determining the proportion of interval estimates that encompass reported statistical benchmarks from the original trials. Additionally, the method may estimate bias in the empirical distributions, providing insight into deviations between synthetic datasets and original trial summaries.

The findings of the method may be presented in tabular and graphical formats to facilitate interpretation. Tables may include point estimates, interval estimates, probabilities for all statistics, coverage percentages for benchmarks, and bias metrics. Graphical outputs may include forest plots depicting point and interval estimates, comparative interval plots highlighting deviations from benchmark values, and other visualizations designed to summarize relationships and variability across trials.

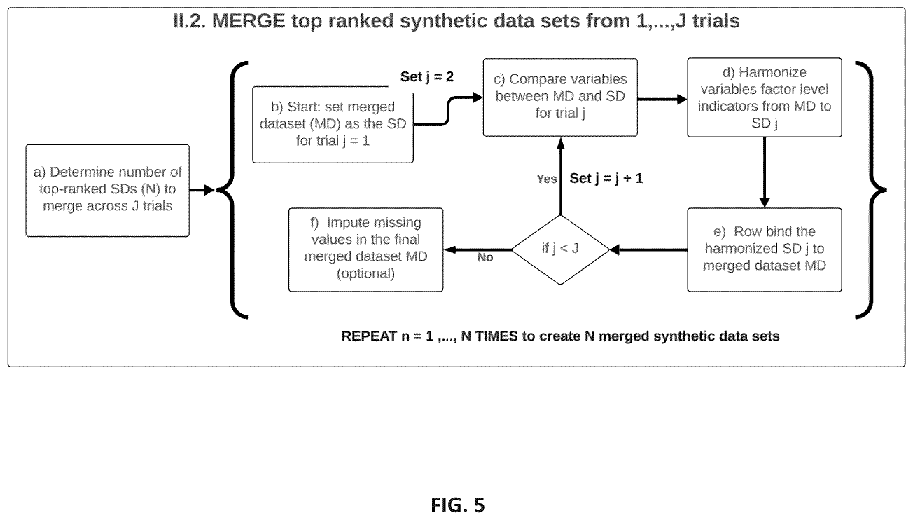

FIG. 5 depicts a flow chart of an example process for merging top-ranked synthetic datasets from a number of trials. The method, in some embodiments, begins by determining the number of top-ranked synthetic datasets, represented as N, and the total number of trials, represented as J. A dynamic variable j is used to facilitate an iterative merging process. Initially, the merged dataset is set as the synthetic dataset for the first trial instance (j=1). Following this initialization, j is incremented to 2, and the method compares variables between the merged dataset and the synthetic dataset for the trial instance j. To harmonize these variables, in some embodiments, factor level indicators (e.g., categorical variable encodings or ranges) from the merged dataset are aligned with the corresponding variables in the synthetic dataset for the trial instance j. The harmonized synthetic dataset for trial instance j is then row-bound to the merged dataset, appending data for trial instance j while preserving consistency across variables. This process of comparison, harmonization, and merging, in some embodiments, is iterated until all trial instances (j) have been processed, where j iterates from 1 through J.

In some embodiments, the harmonization process may involve aligning variable formats, reconciling unit discrepancies (e.g., kilograms vs. pounds for weight), or standardizing missing value encodings. Additionally, logical consistency checks may be performed to ensure that the merged dataset accurately reflects the intended statistical and structural properties of the original datasets.

Once all iterations have been completed and trial instances j have been processed, the method may impute missing values in the final merged dataset. Imputation may involve applying statistical techniques, such as mean or median substitution, regression-based imputation, or probabilistic methods, to fill in gaps while preserving data integrity. The method may also be repeated to create a desired number of merged synthetic datasets. This repetition, in some embodiments, can support downstream processes, such as the creation of distributions of merged datasets for further statistical analysis or machine learning applications.

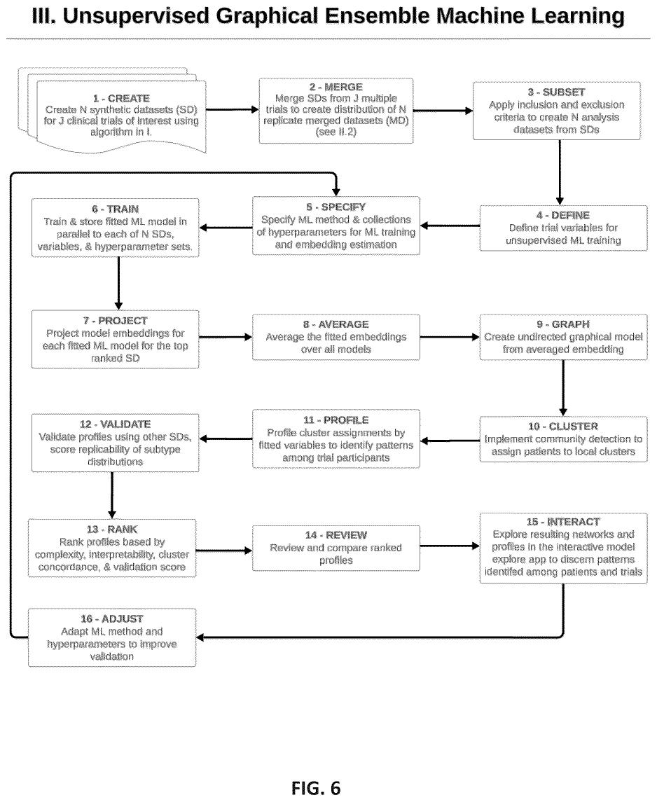

FIG. 6 is a flow chart of an example process for unsupervised graphical ensemble machine learning applied to synthetic datasets. The method, in some embodiments, begins with the creation of a number (N) of synthetic datasets corresponding to a number (J) of clinical trials of interest. These synthetic datasets, in some embodiments, may be generated using the algorithms described in the previous figures. Once created, in some embodiments, the datasets from multiple trials are merged to form a distribution of N replicated merged datasets. Following the merging process, in some embodiments, inclusion and exclusion criteria may be applied to generate N analysis datasets. For example, inclusion criteria might focus on specific demographic subgroups, while exclusion criteria might filter out synthetic patients with inconsistent or incomplete data.

Trial variables, in some embodiments, are then defined for use in unsupervised machine learning training. These variables may include patient-level characteristics, treatment arm assignments, and endpoint results. Additional machine learning methods and collections of hyperparameters for model training and embedding estimation may be specified. These hyperparameters may control aspects of the training process, such as the learning rate, regularization techniques, or dimensions of the embedding space.

The machine learning model, in some embodiments, is trained in parallel across the N synthetic datasets. For each dataset, the model identifies trial variables, applies the specified methods, and tunes hyperparameters to optimize embedding representations. The embeddings for each fitted machine learning model are projected, capturing relationships and patterns among trial variables in a high-dimensional space. Once embeddings are generated for all N datasets, in some embodiments, the embeddings may be averaged to create a composite embedding representation. From the averaged embeddings, in some embodiments, an undirected graphical model is constructed. This model, in some embodiments, encodes relationships between variables as nodes and edges, representing the strength of associations.

Community detection algorithms, in some embodiments, are then applied to the graphical model, assigning patients to local clusters based on shared characteristics. These clusters, in some embodiments, are profiled by examining fitted variables, identifying distinct patterns among trial participants. For example, clusters may reveal subtypes of patients who exhibit similar responses to treatment or share prognostic characteristics. The resulting cluster profiles may be validated using other synthetic datasets to ensure robustness and replicability. Validation may include scoring the replicability of subtype distributions and ensuring that identified patterns are consistent across different datasets. The validated profiles, in some embodiments, are then ranked based on metrics such as complexity, interpretability, cluster concordance, and validation scores. The ranked profiles and associated networks may be reviewed and compared through an automated process implemented by a computer system. Alternatively, or additionally, human analysts may perform the review to discern patterns among patients and trials. The interactive exploration of resulting networks and profiles may facilitate further pattern identification and hypothesis generation.

Adjustments to the machine learning model may also be performed to improve the validation process. These adjustments may involve refining the machine learning methods, modifying the collections of hyperparameters, or enhancing the embedding estimation process. Iterative refinements ensure the model captures meaningful and reproducible relationships among trial variables.

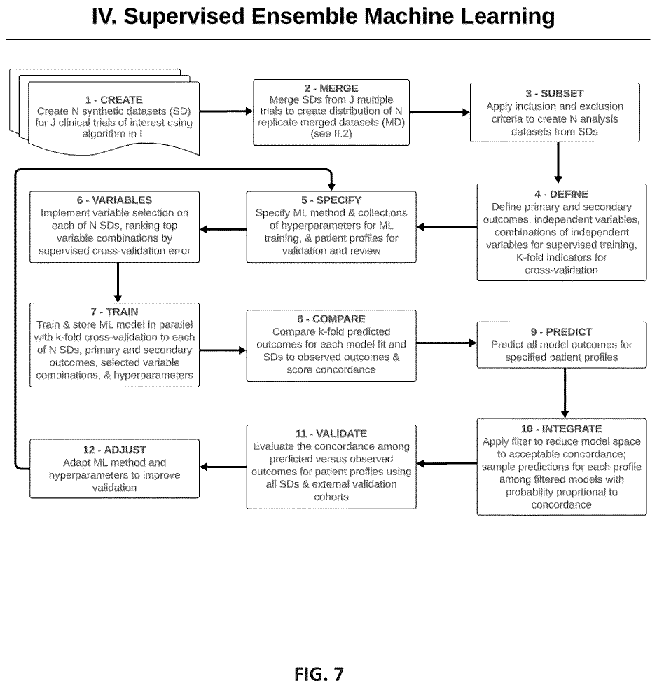

FIG. 7 is a flowchart of an example process for supervised ensemble machine learning. The method, in some embodiments, begins with the creation of a number (N) of synthetic datasets corresponding to a number (J) of clinical trials of interest. These synthetic datasets may be generated using the algorithms described in previous figures. The datasets, in some embodiments, are then merged from the multiple trials to create a distribution of N replicated merged datasets. The merging process is detailed in FIG. 5, wherein trial-specific variables are harmonized to enable cross-trial analysis. Once merged, inclusion and exclusion criteria may be applied to define N analysis datasets. Inclusion criteria may identify specific subgroups or trial arms of interest, while exclusion criteria may remove synthetic data that fails to meet predefined quality thresholds. The analysis datasets, in some embodiments, are then prepared for supervised machine learning by defining the relevant parameters for training.

The parameters may include primary and secondary outcomes, independent variables, and combinations of independent variables for supervised training. Additionally, in some embodiments, k-fold indicators for cross-validation are defined to facilitate robust model evaluation. In preparation for training, in some embodiments, machine learning methods and collections of hyperparameters are specified. These hyperparameters, in some embodiments, may control aspects of the model, such as the learning rate, regularization strength, or tree depth (in tree-based models). Patient profiles for validation and review are also specified, in some embodiments, allowing the model to be tested on representative examples.

Variable selection, in some embodiments, is implemented across each of the synthetic datasets. Top variable combinations are ranked by their supervised cross-validation error, in some embodiments, identifying the combinations most predictive of the primary and secondary outcomes. The machine learning model, in some embodiments, is then trained in parallel for each dataset using the selected variables, primary and secondary outcomes, and specified hyperparameters. k-fold cross-validation, in some embodiments, is employed during training, where the dataset is partitioned into k subsets, and the model is iteratively trained on k−1 subsets and validated on the remaining subset.

Once the models are trained, in some embodiments, the k-fold predicted outcomes for each model fit are compared to the observed outcomes in the synthetic datasets. The resulting concordance, in some embodiments, representing the alignment between predicted and observed outcomes, is scored. This concordance score provides a quantitative measure of model accuracy. Predictions may then be generated for all model outcomes across specified patient profiles. A filtering process is applied to reduce the model space, retaining only models with concordance scores above a predefined threshold. Sample predictions, in some embodiments, are then generated for each profile using the filtered models, with the probability of selecting a specific model being proportional to its concordance score. The concordance of predictions, in some embodiments, is further evaluated by comparing the predictions to observed outcomes for patient profiles across all N synthetic datasets and external validation cohorts.

Adjustments to the machine learning model may also be performed to improve the validation process. These adjustments may involve modifying the machine learning method, refining the hyperparameter space, or updating the variable selection process. Iterative refinements, in some embodiments, allow for improved accuracy, stability, and reliability of the model.

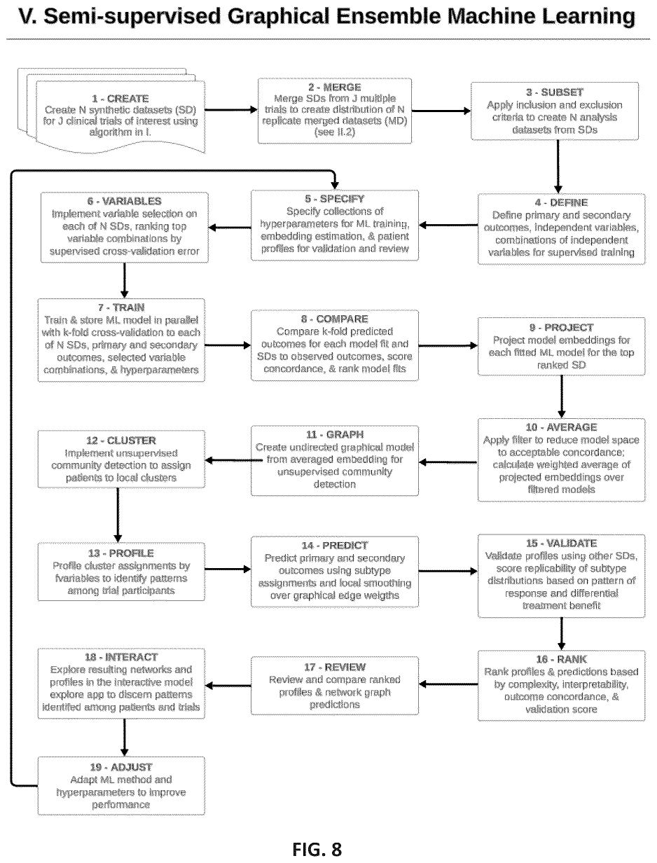

FIG. 8 is a flowchart of an example process of semi-supervised ensemble machine learning. The method, in some embodiments, begins with the creation of a number (N) of synthetic datasets for a number (J) of clinical trials of interest, using the algorithms described in previous figures. These datasets are then merged from the multiple trials to create a distribution of N replicated merged datasets. The merging process is detailed in FIG. 5, facilitating consistency and harmonization of variables across trials. Inclusion and exclusion criteria, in some embodiments, are applied to generate N analysis datasets from the synthetic datasets. For example, inclusion criteria might target specific patient subgroups or trial arms, while exclusion criteria might remove data that does not meet predefined quality standards. Once the datasets are prepared, primary and secondary outcomes, independent variables, and combinations of independent variables for supervised training, in some embodiments, are defined for the merged synthetic datasets.

Embedding estimation and collections of hyperparameters for machine learning training are specified, in some embodiments, as well as patient profiles for validation and review. These parameters may control the configuration of the machine learning models, including regularization, optimization algorithms, and dimensionality of embeddings. Variable selection, in some embodiments, is performed on each of the synthetic datasets, identifying the top combinations of variables based on supervised cross-validation error. These variable combinations, in some embodiments, are ranked to prioritize features most predictive of the outcomes.

The machine learning model, in some embodiments, is trained in concurrently using k-fold cross-validation across the synthetic datasets, with training incorporating the defined primary and secondary outcomes, selected variable combinations, and specified hyperparameters. During training, each dataset is split into k subsets; the model is iteratively trained on k−1 subsets and validated on the remaining subset. This process, in some embodiments, reduces overfitting and improves model reliability. Once trained, in some embodiments, the k-fold predicted outcomes for each model are compared to observed outcomes within the synthetic datasets, and the resulting concordance is scored. Models are ranked based on their concordance scores, reflecting the fit between observed and predicted outcomes. For the top-ranked synthetic datasets, in some embodiments, embeddings from each fitted machine learning model are projected into a shared embedding space. A filter is applied, in some embodiments, to retain only models meeting an acceptable concordance threshold. The weighted average of projected embeddings over the filtered models, in some embodiments, is then calculated, providing a consolidated representation.

An undirected graphical model is constructed, in some embodiments, from the averaged embeddings. This model encodes relationships among variables and patients, which are further analyzed using unsupervised community detection algorithms. The community detection step, in some embodiments, assigns patients to local clusters based on shared characteristics derived from the graphical model. These clusters are profiled by examining fitted variables, revealing patterns and subgroups among trial participants. For example, clusters might represent patients with differential treatment responses or distinct prognostic characteristics. The method may also include predicting primary and secondary outcomes using subtype assignments and local smoothing over graphical edge weights. After profile clusters are assigned, in some embodiments, they are validated using other synthetic datasets to assess the replicability of subtype distributions. Validation may score replicability based on patterns of response and differential treatment benefits. The resulting profiles and predictions, in some embodiments, are ranked based on metrics such as complexity, interpretability, outcome concordance, and validation score. These ranked profiles and associated network graph predictions may be reviewed and compared through an automated process executed by a computer system or manually by human analysts. Additionally, the resulting networks and profiles may be explored in an interactive model exploration application, which enables deeper analysis and pattern identification among patients and trials.

Adjustments to the machine learning model may also be made to improve validation processes. These adjustments may include modifying the specified machine learning methods, refining collections of hyperparameters, or improving embedding estimation processes.

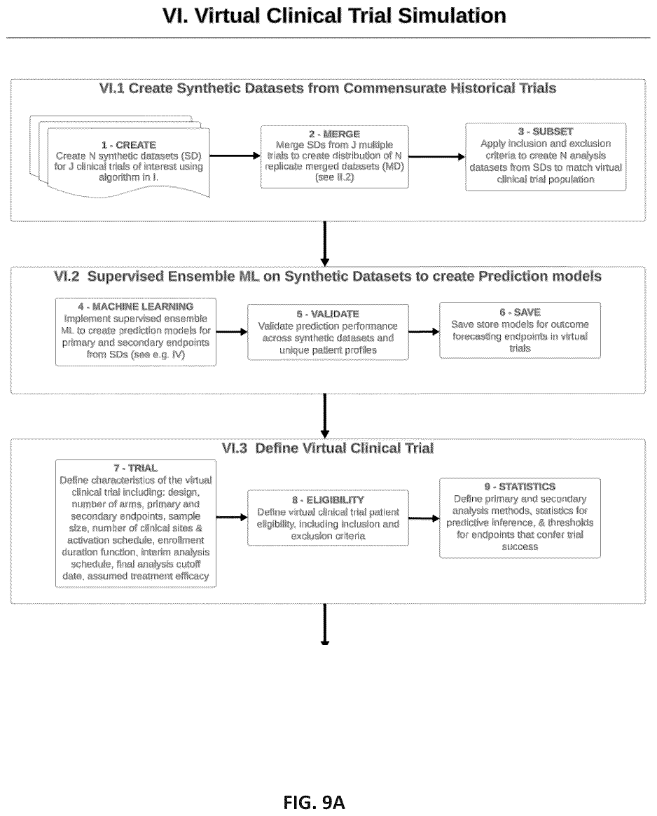

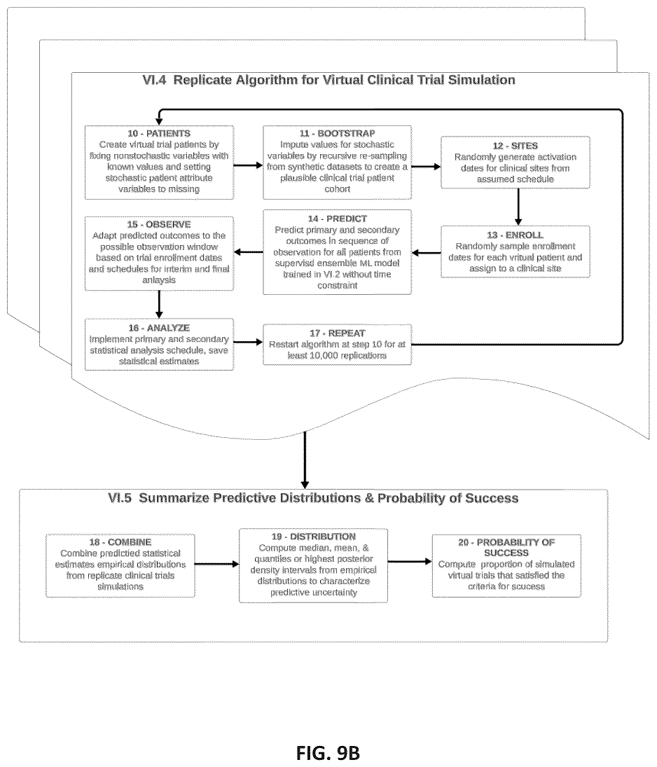

FIG. 9A is a flowchart of an example process for the creation of synthetic datasets from commensurate historical trials, the use of supervised ensemble machine learning to generate prediction models, and the definition of virtual clinical trials.

To begin, in some embodiments, synthetic datasets are created for a number (J) of historical clinical trials of interest using an algorithm, such as the one detailed in FIG. 2. These synthetic datasets, in some embodiments, represent patient-level data reconstructed from statistical summaries and metadata. The synthetic datasets from multiple trials, in some embodiments, are merged to create a distribution of replicate merged datasets. Inclusion and exclusion criteria are applied to generate analysis datasets from the merged synthetic datasets. These criteria, in some embodiments, refine the datasets to align with the population characteristics and eligibility criteria of the virtual clinical trial being simulated. For example, inclusion criteria might specify biomarker-positive patients, while exclusion criteria might eliminate synthetic patients with incompatible demographic or clinical attributes.

Following the preparation of analysis datasets, a machine learning model may be implemented to create prediction models for primary and secondary endpoints. The machine learning ensemble used in this step may employ supervised, unsupervised, or semi-supervised learning techniques, depending on the analysis objectives and dataset characteristics. The prediction models, in some embodiments, are trained on the synthetic datasets to forecast outcomes such as treatment response, adverse events, or progression. Validation of prediction performance may be performed using synthetic datasets and unique patient profiles. Resulting models may then be saved and utilized for forecasting endpoint outcomes in the virtual clinical trials.

The method may also involve defining the characteristics of the virtual clinical trial. This includes specifying key trial design parameters such as the number of arms, primary and secondary endpoints, sample size, number of clinical sites, activation schedules, enrollment duration functions, interim analysis schedules, final analysis cutoff dates, and assumed treatment efficacy. Virtual patient eligibility, in some embodiments, is defined through inclusion and exclusion criteria, ensuring that the virtual cohort aligns with the intended trial design. Additionally, statistical methodologies for primary and secondary analysis, predictive interference, and endpoint thresholds for trial success may be specified.

FIG. 9B depicts a method of replicating virtual clinical trials and summarizing predictive distributions and probabilities of success. The replicate algorithm, in some embodiments, begins by creating virtual trial patients. Non-stochastic variables, such as fixed demographic attributes or known treatment assignments, are assigned predefined values. Stochastic variables, representing attributes subject to variability, in some embodiments, are initially set to “missing” and subsequently imputed through recursive resampling from synthetic datasets. This process, in some embodiments, generates plausible clinical trial cohorts based on the synthetic datasets. The method may include generating activation dates for clinical sites based on predefined schedules. Enrollment dates for each virtual patient are randomly sampled and assigned to clinical sites, simulating staggered enrollment. Primary and secondary outcomes for the virtual patients, in some embodiments, are predicted in sequence of observation using a machine learning model trained without temporal constraints. These predicted outcomes, in some embodiments, are adjusted to fit the observation windows defined by the trial's enrollment dates and analysis schedules. Outcomes may be truncated or otherwise modified to align with interim analyses or the final analysis cutoff.

Statistical analyses for primary and secondary endpoints, in some embodiments, are performed according to pre-specified schedules, and the resulting statistical estimates are saved for each replication. The replicate algorithm, in some embodiments, is then repeated, generating multiple iterations of virtual trials to capture variability and statistical uncertainty. The predictive distributions and probabilities of success, in some embodiments, are then summarized. Predicted statistical estimates from all replicate trials are aggregated into empirical distributions. Summary statistics, including medians, means, quantiles, or highest posterior density levels, in some embodiments, are computed to characterize uncertainty in the trial outcomes. The probability of success, in some embodiments, is calculated as the proportion of replicated virtual trials that meet predefined success criteria, such as achieving endpoint thresholds or specific efficacy metrics.

FIG. 10 illustrates an example system 100 for generating synthetic patient-level data from information reported in a clinical study publication. In some embodiments, system 100 includes a computer system 110 communicatively coupled to a user device 180 and to one or more external data sources 150 via a network 190. In other embodiments, system 100 may include additional components, fewer components, or components arranged in a different manner. The illustrated partitioning into blocks is logical, and any given block may correspond to one or more physical or virtual machines, processes, services, or containers.

In some embodiments, the computer system 110 includes one or more networked computers or one or more processors and memory storing instructions that, when executed by the processors, cause the computer system 110 to perform one or more of the operations described herein. The processors may include general-purpose CPUs, GPUs, or other specialized processing units. The memory may include volatile memory, non-volatile memory, or a combination thereof, and may store executable instructions, configuration parameters, and data structures. In some embodiments, the computer system may include a data ingestion and preprocessing engine 115, a benchmark and constraint engine 120, a stochastic simulation engine 125, an evaluation engine 130, and a data store 135. In some embodiments, each of the engines may correspond to a software module, a group of cooperating services, or a microservice deployed in a containerized environment. In other embodiments, two or more of the engines may share code, state, or hardware resources, or may be integrated into a single application.

In some embodiments, the data ingestion and preprocessing engine 115 may be configured to obtain information describing a clinical study. The information may be received from user device 180, from external data sources 150, or from a combination of both. The information may be obtained in any suitable format, including, for example, electronic documents (such as PDF or word processing files), structured tables, markup formats (such as HTML or XML), or machine-readable data feeds. The data ingestion and preprocessing engine 115 may parse a clinical study publication, identify sections describing trial design, arms, endpoints, and results, and extract values from text, tables, and figures. In some embodiments, the engine 115 may apply natural language processing, pattern matching, optical character recognition, or rule-based parsing to locate trial arms, arm labels, treatment or control assignments, sample sizes, clinical variables, and summary statistics for those variables. In some embodiments, the publication may be provided in a structured format, such as a spreadsheet or a JSON document, and the engine 115 may map fields in the structured format directly into internal data structures.

In some embodiments, the data ingestion and preprocessing engine 115 may also obtain time-to-event information for one or more endpoints of the clinical study. In some embodiments, the time-to-event information may include Kaplan-Meier curve data and corresponding at-risk information extracted from figures or tables in the publication. For example, the engine 115 may read digitized curve coordinates, step changes in survival probability, or tables listing numbers at risk at specified time points. The engine 115 may normalize time units, align reported time points across arms, and map time-to-event information into a representation that associates time indices with survival probabilities and at-risk counts for each trial arm. In some embodiments, the engine 115 may construct, for each trial arm, a per-arm data structure that specifies an arm label, a treatment or control assignment, a sample size, one or more clinical variables, and reported distributions for the clinical variables, such as means, medians, standard deviations, ranges, or proportions.

In some embodiments, the benchmark and constraint engine 120 operates on the preprocessed information produced by the engine 115. The benchmark and constraint engine 120 may determine a set of benchmarks that may include reported statistical values from the clinical study and data specifying one or more statistical analysis methods used to produce the reported statistical values. In some embodiments, the benchmarks may include hazard ratios and associated confidence intervals for one or more endpoints, response or event rates for trial arms or subgroups, survival probabilities or median time-to-event values at specified time points, and summary statistics for baseline covariates. In some embodiments, the benchmark and constraint engine 120 stores the benchmarks in a data structure that associates each reported statistical value with a description of the analysis method used, the variables involved, and any subgroup definitions.

In some embodiments, the benchmark and constraint engine 120 may determine a set of constraints based on the set of benchmarks and the obtained information describing the clinical study. The constraints may include numerical limits on synthetic values for clinical variables, such as ranges for means, proportions, or hazard ratios consistent with reported values and confidence intervals. The constraints may also include clinical logical rules specifying permitted temporal and clinical relationships among events, such as rules requiring that treatment start precede treatment-dependent events, rules preventing progression or response events from occurring after a death event for a participant, and rules requiring censoring times and follow-up windows to be consistent with reported follow-up information. In some embodiments, constraint definitions are stored in the data store 135 as parameterized rules that can be evaluated against candidate synthetic patient records. In some embodiments, constraints may be encoded as functions or predicates that accept candidate values and return indications of whether those values satisfy the constraints.

In some embodiments, the stochastic simulation engine 125 may be configured to generate candidate synthetic patient-level datasets that are constrained by the benchmarks and constraints determined by the constraint engine 120. The stochastic simulation engine 125 may initialize a stochastic simulation using a randomized seed, which may be stored for reproducibility, and an initial candidate dataset. The initial candidate dataset may be constructed by sampling values for clinical variables from distributions that approximate reported univariate distributions, by assigning participants to arms based on sample sizes, and by assigning initial event times using a rough approximation to the Kaplan-Meier curves. In some embodiments, the initial candidate dataset may be an empty dataset or a dataset constructed using heuristic rules.

During operation, the stochastic simulation engine 125 may, for each arm of the clinical study, propose a candidate set of synthetic patient records. In some embodiments, the stochastic simulation engine 125 selects synthetic values for clinical variables using randomized sampling from per-arm distributions that have been calibrated to the reported means, medians, standard deviations, or proportions, and then adjusts or rejects sampled values that would violate one or more constraints. The stochastic simulation engine 125 may assign time-to-event and censoring indicators for each synthetic patient based on the mapped Kaplan-Meier information, such as step changes in survival probability and at-risk counts, and on the discrete time intervals used to represent the time axis. In some embodiments, the stochastic simulation engine 125 may construct multivariable synthetic profiles for participants by sampling correlated variables jointly, using correlation structures inferred from published data or from prior knowledge. In some embodiments, the stochastic simulation engine 125 may treat variables as independent at the sampling stage and allow dependencies to emerge through constraint enforcement and iterative adjustment.

In some embodiments, the stochastic simulation engine 125 may assemble per-arm synthetic records into one or more whole-trial candidate datasets. A whole-trial candidate dataset may include synthetic records for all arms of the study, each record including clinical variables, arm assignment, and event-time and censoring indicators. The stochastic simulation engine 125 may generate a single whole-trial candidate dataset or may generate a plurality of whole-trial candidate datasets over multiple iterations or simulation runs. In some embodiments, the stochastic simulation engine 125 operates in a distributed or parallel fashion, with different processing threads or nodes proposing synthetic records for different arms, different subsets of participants, or different candidate datasets concurrently.

The evaluation engine 130 is configured, in some embodiments, to analyze candidate datasets produced by the stochastic simulation engine 125. The evaluation engine 130 may execute, on a whole-trial candidate dataset, one or more statistical analysis methods corresponding to the set of benchmarks. For example, the evaluation engine 130 may fit Cox proportional hazards models to compute hazard ratios and confidence intervals for time-to-event endpoints, compute response or event rates for specified subgroups, compute summary statistics for baseline covariates, and compute survival probabilities at specified time points. In some embodiments, the evaluation engine 130 may then compare the computed statistical values to the reported statistical values in the benchmark set to determine one or more deviation scores. The deviation scores may be based on absolute differences, relative differences, squared differences, differences on a transformed scale, or other distance measures.

In some embodiments, the evaluation engine 130 may determine a bias metric associated with a whole-trial candidate dataset based on the deviation scores and information indicating whether the set of constraints is satisfied for the dataset. The bias metric may aggregate deviations across multiple benchmarks and may incorporate penalties or increased contributions when constraints are not satisfied. The evaluation engine 130 may further determine a probability value from the bias metric and from constraint satisfaction information. The probability value may influence how the stochastic simulation engine 125 treats the whole-trial candidate dataset, such as whether the dataset is retained without change, whether selected records or variables are modified through targeted adjustment, whether the dataset is replaced with a newly proposed dataset, or whether the simulation transitions to another candidate dataset. In some embodiments, the evaluation engine 130 and the stochastic simulation engine 125 operate together as a feedback loop, iteratively proposing, evaluating, and adjusting candidate datasets.

In some embodiments, the data store 135 may represent one or more storage components accessible to the computer system 110. In some embodiments, the data store 135 includes a relational database, a key-value store, a file system, an object store, or a combination thereof. The data store 135 may store ingested clinical study publications, structured per-arm data and trial representations, benchmark sets and associated analysis definitions, constraint sets, intermediate whole-trial candidate datasets, deviation scores, bias metrics, probability values, and selected synthetic datasets that satisfy predefined quality criteria. The data store 135 may also store configuration information such as user-specified tolerances for acceptable deviation, weights assigned to different benchmark statistics, random seeds associated with simulation runs, and logging information useful for auditing or reproducing synthetic dataset generation.

In some embodiments, the user device 180 may be any computing device capable of communicating with computer system 110, such as a laptop computer, desktop computer, tablet, or smartphone. In some embodiments, the user device 180 executes a client application or web browser that provides a graphical user interface or an application programming interface for interacting with the computer system 110. In some embodiments, a user operating the user device 180 may upload clinical study publications, select which reported statistics to treat as benchmarks, specify tolerances or weights for different benchmarks, configure simulation parameters, request generation of synthetic datasets, and view or download resulting synthetic datasets and analyses. In some embodiments, the user device 180 may act as a system-to-system client that programmatically invokes services provided by the computer system 110.

In some embodiments, the external data sources 150 may include one or more repositories that store clinical study publications or related data. In some embodiments, the external data sources 150 may include journal repositories, preprint servers, clinical trial registries, regulatory document repositories, sponsor-operated data portals, or combinations of these. The computer system 110 may communicate with the external data sources 150 via the network 190 to retrieve publications, structured summaries, or metadata that include information used by the data ingestion and preprocessing engine 115. In some embodiments, the external data sources 150 may provide application programming interfaces that allow the computer system 110 to query for specific studies, endpoints, or populations.

In some embodiments, the network 190 may include any combination of one or more communication networks that support data exchange among the user device 180, external data sources 150, and the computer system 110. The network 190 may include public networks, private networks, wired networks, wireless networks, or combinations thereof. In some embodiments, communication over the network 190 may employ encrypted channels, virtual private networks, or other security mechanisms. In other embodiments, one or more of the user device 180, external data sources 150, and the computer system 110 may be co-located, and communication among them may occur over a local interconnect rather than a wide area network. In some embodiments, components of the computer system 110 may themselves be distributed across multiple physical locations and may communicate using the network 190.

Although specific components are illustrated in FIG. 10, the depicted arrangement is one non-limiting example. In some embodiments, one or more of the engines shown in FIG. 10 may be omitted, combined, or replaced by alternative components that perform similar functions. For example, the functionality attributed to the benchmark and constraint engine 120 may be performed by a generalized analysis engine that also handles evaluation tasks, or the functionality of the stochastic simulation engine 125 may be distributed across several services specializing in proposal generation, constraint checking, and dataset assembly. Similarly, the data store 135 may be implemented as multiple logical data stores, such as separate stores for source publications and for synthetic datasets. The techniques described herein may be implemented using other architectures that differ from the specific example shown in FIG. 10 while still providing generation and evaluation of synthetic patient-level datasets from information reported in clinical study publications.

FIG. 11 illustrates an example method 200 for generating simulated patient records based on information reported in a clinical study publication. In some embodiments, the method 200 is performed by the computer system 110 of FIG. 10, by one or more processors executing instructions stored in memory. In other embodiments, some operations of the method 200 may be performed by the user device 180, by a distributed computing environment, or by a combination of systems. The method 200 is shown as a sequence of discrete blocks for purposes of explanation, but the illustrated ordering is not required, and two or more of the operations may be performed in parallel, in a different order, or as part of a larger workflow.

In some embodiments, the method 200 may include obtaining a clinical study publication 210. The computer system 110 may obtain a publication that describes a clinical trial or other clinical study from the external data sources 150, from the user device 180, or from both. The publication may be received in electronic form as a document file, as a structured data feed, as an image file, or as a combination of formats. The publication may comprise text sections describing trial design, inclusion and exclusion criteria, outcome definitions, and statistical methods, as well as tables reporting baseline characteristics and results, and figures showing time-to-event curves. In some embodiments, the publication is a peer-reviewed journal article. In other embodiments, the publication may be a conference abstract, a regulatory review document, a sponsor-generated study report, or a preprint. The computer system 110 may store the clinical study publication in the data store 135 along with metadata such as a study identifier, disease area, and publication source.

In some embodiments, the method 200 may include generating simulated patient records 215. The computer system 110 may parse the clinical study publication obtained at block 210 and construct an internal representation of the reported study, including trial arms, arm labels, sample sizes, clinical variables, and time-to-event information. The computer system 110 may then use this internal representation as a basis for generating synthetic or simulated records, each record corresponding to a simulated participant in the study. In some embodiments, generating simulated patient records includes sampling clinical variable values from distributions calibrated to reported univariate statistics, such as sampling age values to match a reported mean and standard deviation for age in a particular arm, or sampling a binary variable to match a reported proportion. In other embodiments, generating simulated patient records may include sampling multivariable profiles using correlation structures, assigning treatment or control labels consistent with reported sample sizes for each arm, and assigning event-time and censoring indicators based on time-to-event information extracted from Kaplan-Meier curves and at-risk tables. The result of block 215 may be a collection of simulated patient records that approximates the reported characteristics of the clinical study but does not reuse or require any individual patient-level data from the original trial.

In some embodiments, the method 200 may include adjusting the simulated patient records 220. The computer system 110 may evaluate whether the simulated records generated at block 215 conform to numerical bounds and clinical logical rules. For example, the computer system 110 may compare aggregate statistics computed from the simulated records, such as means, proportions, hazard ratios, or survival probabilities, to corresponding reported statistics from the clinical study publication. The computer system 110 may also evaluate logical relationships among events for each simulated participant, such as verifying that a death event does not occur before treatment assignment, or that a progression event does not occur after a death event. When the simulated records do not conform to one or more numerical or logical conditions, the computer system 110 may adjust the simulated records by resampling values for selected variables, modifying event times for selected participants, adjusting censoring times, or applying other transformation rules that move the simulated dataset closer to the reported behavior. In some embodiments, adjustments may be targeted to variables and endpoints that exhibit larger discrepancies, whereas in other embodiments adjustments may be applied uniformly across variables.

In some embodiments, the method 200 may include scoring the simulated patient records 225. The computer system 110 may execute one or more statistical analyses on the simulated dataset that corresponds to analyses reported in the clinical study publication. For example, the computer system 110 may compute endpoint-specific hazard ratios and confidence intervals using Cox proportional hazards models, compute response rates or event rates for trial arms or subgroups, or compute summary statistics for baseline covariates. The computer system 110 may then compare the statistics computed from the simulated records to the corresponding reported values and determine one or more scores that quantify deviation or bias. The scores may reflect absolute differences, relative differences, squared differences, or other distance measures, and may be aggregated across multiple endpoints or variables to form a composite score or bias metric. In some embodiments, the score associated with a simulated dataset also reflects whether numerical and logical constraints are satisfied for the simulated records.

In some embodiments, the method 200 may include selecting a subset of the simulated patient records 230. The computer system 110 may use the scores determined at block 225 to assess the quality of the simulated dataset or of individual simulated records. When multiple simulated datasets are generated, the computer system 110 may rank the datasets by their scores and select one or more datasets with scores that are within an acceptable range. When a single simulated dataset is generated, the computer system 110 may select a subset of simulated records whose inclusion best preserves concordance with the reported statistics. In some embodiments, the selection at block 230 is driven by a probabilistic rule that depends on the score or bias metric and on indications of constraint satisfaction, such that simulated datasets with better concordance are more likely to be selected. In other embodiments, the selection may be based on deterministic thresholds, such as retaining only those datasets whose scores fall below a tolerance level.

In some embodiments, the method 200 may include storing the simulated patient records 235. The computer system 110 may store a selected simulated dataset, or a subset of simulated records, in the data store 135 as a synthetic representation of the clinical study. The stored data may include, for each simulated participant, values for clinical variables, arm assignment, and event-time and censoring indicators, as well as associated scores or metadata indicating how the dataset was generated. In some embodiments, the stored simulated records are used as inputs to additional processes, such as statistical modeling, meta-analysis across multiple studies, machine learning model training, or virtual trial simulation. In other embodiments, the simulated records may be exported from the system 100 to other systems, stored in external repositories, or provided to users through the user device 180. The method 200 may be repeated for multiple clinical study publications, and the data store 135 may thus contain synthetic datasets corresponding to multiple trials that can be analyzed individually or in combination.

FIG. 12 illustrates an example method 300 for generating a synthetic dataset from a clinical study publication. In some embodiments, the method 300 may include obtaining a clinical study publication 310 and mapping time-to-event information reported in the publication to discrete time intervals 315, determining per-arm data structures that capture reported distributions for clinical variables and outcomes 320, and assembling the per-arm data structures into a trial representation that records temporal parameters and associates clinical variables with corresponding endpoints 325. The method 300 may further include determining a set of benchmarks and a set of constraints 330 based on reported statistical values and clinical logical rules, proposing a candidate set of synthetic patient records for each arm of the trial 335 using randomized sampling subject to the set of constraints, and combining the proposed records into a whole-trial candidate dataset. In some embodiments, the method 300 may include executing, on the whole-trial candidate dataset, one or more statistical analyses specified by the benchmarks 340, determining one or more deviation scores and a bias metric that quantify concordance between computed statistics and reported statistics 345, and determining a probability value 350 that influences whether the whole-trial candidate dataset is retained, modified through targeted adjustment, replaced with a newly proposed dataset, or transitioned to another candidate dataset. The method 300 may include storing a selected synthetic dataset 355 in a data store and may be performed iteratively by the computer system 110 to generate and evaluate multiple candidate datasets until one or more datasets satisfy predefined quality criteria.

In some embodiments, the method 300 may include obtaining a clinical study publication 310. The computer system 110 may obtain a publication that describes a clinical trial or other clinical study from the external data sources 150, from the user device 180, or from both. The publication may be received in electronic form as a document file, a structured data export, markup content, or an image-based representation, and may include text, tables, and figures describing trial design, arms, eligibility criteria, endpoints, statistical methods, and reported results. In some embodiments, the publication comprises a peer-reviewed journal article or conference paper. In other embodiments, the publication may comprise a conference abstract, a poster, a regulatory review document, a sponsor-prepared clinical study report, a preprint, or an entry in a clinical trial registry. The computer system 110 may store the clinical study publication in the data store 135 together with metadata such as a study identifier, disease area, publication date, and source repository, and may associate multiple publications that relate to the same underlying clinical study.

In some embodiments, the method 300 may include obtaining the clinical study publication 310 in an environment in which patient-level data from the underlying clinical study is not available to the computer system 110. For example, the computer system 110 may be configured to operate based solely on information that is reported in one or more publications and does not require access to individual records for participants enrolled in the trial. As used herein, patient-level data may refer to data in which each record corresponds to a specific participant and includes one or more fields that allow that participant's measurements to be associated across time or across variables, such as a subject identifier, visit identifiers, treatment assignment flags, baseline covariates, and event-time and censoring indicators. In contrast, many clinical study publications provide only aggregate or summary information, such as per-arm sample sizes, arm labels, proportions for categorical variables, summary statistics for continuous variables, hazard ratios with confidence intervals, response rates, and time-to-event information represented as Kaplan-Meier curves and at-risk tables, without exposing individual participant identifiers or per-participant records. In some embodiments, the computer system 110 may treat the publication as the sole source of clinical study information and may generate synthetic patient-level data that is concordant with the reported summary statistics while remaining independent of any actual patient-level records. In other embodiments, the computer system 110 may be configured to use patient-level data when it is available, but the operations described for the method 300 may still be performed using only publication-level information in scenarios where patient-level data cannot be accessed due to regulatory, privacy, or contractual restrictions.

In some embodiments, the method 300 may include mapping time-to-event information 315. The computer system 110 may obtain time-to-event information from the clinical study publication, such as Kaplan-Meier curve data and corresponding at-risk information for one or more trial arms. The Kaplan-Meier curve data may be available as digitized curve coordinates, as tabulated survival probabilities at specified time points, as a combination of both, or as an image that is processed to extract curve points. The at-risk information may be provided as tables indicating, for each arm, the number of participants at risk at a sequence of reported time points. The computer system 110 may define, for each arm, a discrete set of time points that form a time grid and may map the reported Kaplan-Meier and at-risk information onto that time grid so that survival probabilities and at-risk counts are associated with specific intervals on the grid.

In some embodiments, the method 300 may include determining calibrated event-time and censoring indicators based on the mapped time-to-event information 315. The computer system 110 may, for each interval on the time grid and for each arm, estimate how many participants experience an event and how many participants are censored within that interval, using changes in survival probability and at-risk counts between consecutive time points as guidance. For example, when the Kaplan-Meier curve shows a decrease in survival over a particular interval, and the at-risk table shows a reduction in the number at risk, the computer system 110 may attribute a portion of that reduction to event occurrences and a portion to censoring, subject to consistency with the reported survival function. The computer system 110 may use these interval-level event and censor counts as targets that later guide assignment of event times and censoring times for synthetic participants, so that aggregated synthetic event patterns align with the published curves.

In some embodiments, the method 300 may include constructing internal time-to-event structures that encode the mapped time-to-event information 315 for use in subsequent operations. The computer system 110 may, for each trial arm, store the time grid, the mapped survival probabilities, the mapped at-risk counts, and the calibrated interval-level event and censor counts in a per-arm data structure that can be accessed by other components. These structures may support generation of synthetic event-time and censoring indicators by allocating individual synthetic participants to event or censor categories within each interval in proportions consistent with the calibrated counts. In other embodiments, the computer system 110 may represent the time-to-event information using a continuous-time model, such as a piecewise-constant hazard representation or a parametric survival model fitted to the mapped Kaplan-Meier data and may use that representation in place of or in addition to the discrete time grid when assigning event times and censoring times to synthetic participants.

In some embodiments, the method 300 may include determining per-arm data structures 320. The computer system 110 may, for each arm identified in the clinical study publication obtained at block 310, construct a structured representation that records arm-specific information. The structured representation for an arm may include an arm label, an indication of whether the arm is a treatment arm or a control arm, a sample size for the arm, and one or more clinical variables for which summary information is reported. For each clinical variable, the per-arm data structure may store one or more reported distributional quantities, such as a mean, a standard deviation, a median, a range, or a proportion, and may store categorical levels and counts for categorical variables such as gender, biomarker status, or comorbidity indicators. The per-arm data structure may further include outcome-related quantities for the arm, such as survival rates at specified time points, median overall survival, median progression-free survival, response rates, or other endpoint summaries, and may maintain references to the time-to-event information mapped at block 315, including the time grid, mapped survival probabilities, at-risk counts, and interval-level event and censor allocations for the arm.

In some embodiments, determining per-arm data structures 320 may include populating those structures using information obtained from the clinical study publication by manual entry, automated extraction, or a combination of both. For example, a user operating the user device 180 may review a PDF version of the publication and enter values for arm labels, sample sizes, baseline characteristics, and endpoint summaries into a user interface, and the computer system 110 may encode those values into the per-arm data structures. In other embodiments, the computer system 110 may apply optical character recognition techniques, large language models, table parsers, or rule-based extractors to identify and extract numerical values and labels from text, tables, and figures in the publication. In some embodiments, specialized tools, such as plot-digitization utilities, may be used to extract coordinates of Kaplan-Meier curves from published graphs, and the extracted coordinates may be associated with the corresponding arm-level data structures. The per-arm data structures may be stored in the data store 135 as arm-level objects having fields for identifiers, clinical variables, distribution parameters, endpoint summaries, and links to time-to-event representations, and may be used as inputs to subsequent simulation and evaluation operations.

In some embodiments, determining per-arm data structures 320 may also include associating each arm-level structure with trial-level context and computational parameters that are used in later stages of the method 300. For example, the per-arm data structures may record or reference trial phase information, treatment mechanism of action, treatment schedule, follow-up duration, enrollment windows, and data cutoff dates that are reported in the clinical study publication, so that these parameters can be taken into account when generating synthetic patient records. In some embodiments, the computer system 110 may construct or update per-arm data structures in a parallel or distributed manner, for instance by assigning different arms or different subsets of variables to separate processing threads, CPU cores, or accelerator devices such as graphics processing units, tensor processing units, or other AI accelerators. The level of detail in the per-arm data structures and the degree of concurrent processing may be adjusted based on the amount of available computing resources, allowing the method 300 to scale to studies with many arms, many clinical variables, or a large number of replicated synthetic datasets.

In some embodiments, the method 300 may include assembling the per-arm data structures into a trial representation 325. The computer system 110 may construct a trial-level object or data structure that aggregates the per-arm data structures determined at block 320 and organizes them together with trial-level metadata obtained from the clinical study publication. The trial representation may include identifiers for the clinical study, indications of trial phase (for example, Phase I, II, or III), the therapeutic area, the mechanism of action of the investigational treatment, and information about the overall trial design, such as the number and type of arms, randomization ratios, stratification factors, follow-up duration, treatment cycles, and planned or actual data cutoff dates. In some embodiments, the trial representation also includes references to the time-to-event structures produced at block 315 so that each arm-level structure is linked to a corresponding time grid, mapped survival probabilities, and at-risk counts used in time-to-event modeling. The computer system 110 may store the trial representation in the data store 135 as a trial-level object that references the individual per-arm data structures and associated time-to-event structures and may access the trial representation when performing subsequent operations of the method 300. In some embodiments, the trial representation may be implemented as a trial object that is instantiated in software with one or more arm objects (for example, treatment and control arms) and fields that encode temporal parameters, design attributes, indication information, and external references, providing a concrete, machine-readable instantiation of the clinical trial for downstream processing.

In some embodiments, assembling the per-arm data structures into the trial representation 325 may include establishing associations between clinical variables, arms, and endpoints and recording configuration parameters that apply across arms. The trial representation may record which clinical variables are measured at baseline, which variables are time-varying, and which variables are used as covariates in reported statistical analyses and may include mappings that associate particular variables or combinations of variables with specific endpoints such as overall survival, progression-free survival, response, or composite outcomes. The trial representation may further record subgroup definitions used in reported analyses, for example age-defined subgroups, biomarker-defined subgroups, or prior-therapy-defined subgroups, and may include one or more analysis definitions, each specifying an endpoint, a model type or statistical method, the variables involved, and arm comparisons of interest. In some embodiments, the trial representation also includes parameters specifying the discrete time grid to be used for simulation and analysis, tolerances for matching reported statistics, weights assigned to different benchmarks when computing bias metrics, and options for how constraints are enforced during synthetic data generation. Portions of the trial representation may be maintained in memory during processing, and different components of the computer system 110 may access subsets of the trial representation concurrently in a distributed or parallel computing environment when determining the set of benchmarks and constraints at block 330 and when generating, evaluating, and storing synthetic datasets in later stages of the method 300. In some embodiments, the trial representation corresponds to a trial object that includes fields specifying a trial phase (for example, a Phase III randomized open-label study), a treatment line (for example, second-line therapy), a disease indication (for example, metastatic non-small cell lung cancer with an adenocarcinoma histological subtype), a therapeutic class (for example, chemotherapy), and provenance and reference metadata (for example, a creator identifier, a descriptive summary, a digital object identifier for a publication, and a registry identifier), thereby providing a concrete example of how trial-level metadata is encoded in a structured format.

In some embodiments, the method 300 may include determining a set of benchmarks and a set of constraints 330. The computer system 110 may determine a set of benchmarks from the trial representation assembled at block 325 and from the clinical study publication obtained at block 310. The set of benchmarks may comprise reported statistical values together with information indicating the analyses from which those values were produced. For example, the set of benchmarks may include arm-level or subgroup-level summary statistics for baseline covariates, endpoint-specific hazard ratios with corresponding confidence intervals, response or event rates, survival probabilities at specified time points, or median time-to-event values. In some embodiments, each benchmark entry in the set may include fields identifying the variables involved, the endpoint to which the benchmark pertains, the arms or subgroups being compared, and a description of the statistical method used, such as a Kaplan-Meier estimator, a Cox proportional hazards model, a logistic regression, or a chi-squared comparison of proportions.

In some embodiments, determining the set of benchmarks 330 may include selecting which reported statistics from the clinical study publication are to be treated as benchmarks and, optionally, incorporating benchmarks from external sources. A user operating the user device 180 may specify, via a user interface, that particular reported values are to be used as benchmarks, such as overall survival hazard ratios for specific treatment versus control comparisons, progression-free survival curves for biomarker-defined subgroups, or response rates for arms defined by prior lines of therapy. In other embodiments, the computer system 110 may automatically identify candidate benchmarks by scanning the trial representation for reported statistics that satisfy preconfigured criteria, such as statistics labeled as primary or key secondary endpoints, statistics accompanied by confidence intervals, or statistics that can be recomputed from synthetic data using available analysis definitions. In some embodiments, the set of benchmarks may further incorporate external comparison values, such as summary statistics from real-world datasets or from other published trials and may record mappings that relate variables in the synthetic trial representation to variables in external benchmark datasets.

In some embodiments, the method 300 may include determining a set of constraints 330 based on the set of benchmarks and the obtained information describing the clinical study. The set of constraints may encode numerical conditions and clinical logical rules that the synthetic patient-level data is expected to satisfy. Numerical constraints may specify permissible ranges or tolerances for synthetic statistics when compared to the benchmarks, such as requiring that synthetic means or proportions for baseline variables fall within a specified distance of reported values, that synthetic hazard ratios lie within a tolerance band around reported hazard ratios or within reported confidence intervals, or that synthetic survival probabilities at particular time points differ from the reported Kaplan-Meier estimates by less than a threshold. In some embodiments, the constraints may be represented as parameterized inequalities, tolerance intervals, or functions that accept synthetic statistics as inputs and return indications of whether the statistics are acceptable relative to the benchmarks.

In some embodiments, the set of constraints 330 may also include clinical logical rules that restrict the relationships among events, times, and variables at the level of individual synthetic participants. These rules may include, for example, requirements that treatment assignment for a synthetic participant occurs at or before a baseline time, that time-to-event values and censoring times are non-negative and ordered in time for each participant, that a death event for a participant does not occur before a progression, response, or other intermediate event associated with that participant, and that censoring times occur on or before the last recorded event time and within a follow-up window consistent with the trial design. Additional rules may require that all records for a synthetic participant remain associated with a single trial arm and a consistent treatment or control assignment, that event counts aggregated over the discrete time grid match calibrated interval-level event and censor counts determined at block 315 within specified tolerances, or that subgroup definitions used in benchmarks (such as biomarker-positive subsets) are applied consistently when computing synthetic statistics. In some embodiments, the constraints are stored in the data store 135 as a constraint set associated with the trial representation and are subsequently evaluated when proposing candidate synthetic patient records at block 335 and when determining bias metrics and probability values at blocks 345 and 350.

In some embodiments, the method 300 may include proposing a candidate set of synthetic patient records 335. The computer system 110 may implement a stochastic simulation that progresses through successive states of a Markov chain or Markov-like process in which each state corresponds to a synthetic dataset representing the clinical study. The simulation may be initialized at a new state, for example at state t+1, starting from an empty dataset or from an initial candidate dataset constructed using heuristic sampling of clinical variables and event times. For a given iteration, the computer system 110 may, for each trial arm represented in the trial object assembled at block 325, generate a candidate arm-level dataset using probabilistic models or other computational methods. The candidate arm-level dataset for an arm may include imputed or sampled values for clinical variables, arm assignments, and event-time and censoring indicators, and may be constrained to be consistent with the distributions, time-to-event structures, and constraints determined at blocks 315, 320, and 330.

In some embodiments, the method 300 may include performing a per-arm proposal process that refines candidate arm-level datasets until univariate distributions are aligned with reported statistics. For example, the computer system 110 may initially impute missing values and sample clinical variable values for an arm so that univariate distributions, such as the distributions of patient age, biomarker positivity, or comorbidity indicators, approach the reported means, medians, standard deviations, ranges, or proportions specified in the per-arm data structures. When the simulated univariate distributions for one or more variables fail to reach specified tolerances relative to the benchmarks and numerical constraints, the computer system 110 may, in some embodiments, restart the proposal step for that arm by discarding the current candidate arm-level dataset and resampling values under updated random seeds or adjusted sampling parameters. Once the univariate distributions for the arm are within acceptable tolerances, the per-arm proposal process may proceed to construct multivariable synthetic profiles.

In some embodiments, the method 300 may include generating multivariable synthetic patient profiles and aligning those profiles with reported endpoint results as part of the proposal step 335. Each synthetic patient profile for a given arm may include demographic characteristics, baseline biomarker levels, comorbidity indicators, and a treatment or control assignment, together with candidate event-time and censoring indicators allocated using the time-to-event structures mapped at block 315. The computer system 110 may compute one or more severity scores or risk scores for synthetic patients based on their characteristics and may use those scores to influence assignment of events or outcomes such as progression, response, or survival, so that aggregated endpoint results for the arm resemble reported progression-free survival, overall survival, or response rates. When the candidate profiles fail to align with endpoint results or surrogate outcomes, such as intermediate progression markers or adverse event metrics, the computer system 110 may iterate the proposal process, resampling or adjusting selected variables or event times for a subset of synthetic patients until arm-level endpoint summaries are consistent with the benchmarks and constraints. In some embodiments, convergence checks are performed at the arm level, and when convergence criteria are satisfied, the method 300 may proceed to propose candidate records for the next trial arm.

In some embodiments, the method 300 may include assembling the candidate arm-level datasets generated during the proposal step 335 into a whole-trial candidate dataset that captures all arms of the clinical study for the current simulation state. The whole-trial candidate dataset may be treated as an emulated dataset that plausibly could have produced the reported trial analyses, given that it is constructed based on summary statistics, Kaplan-Meier curves, and constraints that encode clinical relationships such as that patients do not experience progression events after a death event or that risk of certain comorbid conditions increases with age. The constraints, including those obtained from the publication and those reflecting knowledge about clinical plausibility, may guide the search over the space of possible whole-trial candidate datasets and influence which candidate datasets are retained or further refined. In some embodiments, the search is governed by an objective function that accounts for the benchmarks and constraints and may be non-differentiable, such that techniques that rely on gradients, such as gradient descent, are not applicable. In some embodiments, the stochastic simulation may execute a large number of iterations, for example more than ten thousand iterations or more than one million iterations, as it proposes and refines candidate datasets across trial arms to obtain one or more whole-trial candidate datasets that exhibit acceptable concordance with reported trial results.

In some embodiments, the method 300 may include executing one or more statistical analyses 340. The computer system 110 may, for a whole-trial candidate dataset assembled at block 335, identify analysis definitions associated with the set of benchmarks determined at block 330 and apply corresponding statistical methods to the synthetic patient records. For example, for time-to-event endpoints, the computer system 110 may reconstruct Kaplan-Meier survival curves from the synthetic dataset, compute survival probabilities at specified time points, estimate median time-to-event values, or fit Cox proportional hazards models or other survival models to obtain hazard ratios and associated confidence intervals for specified arm comparisons or subgroups. For binary or categorical endpoints, the computer system 110 may compute response rates or event rates for arms or subgroups and may fit generalized linear models such as logistic regression models to estimate odds ratios or risk ratios. For continuous variables, the computer system 110 may compute means, medians, standard deviations, ranges, or quantiles by arm or subgroup. The analyses executed at block 340 may thus replicate, on the synthetic dataset, the analyses that produced the reported statistics identified as benchmarks.

In some embodiments, executing statistical analyses 340 may include fitting multivariable or model-based analyses specified in the benchmark definitions and recording the resulting model parameters as part of the synthetic analysis results. For instance, the computer system 110 may fit multivariate regression models, generalized linear models, or other parametric or semi-parametric models that incorporate clinical covariates, stratification factors, or interaction terms described in the clinical study publication. In other embodiments, the computer system 110 may apply machine learning-based methods, such as gradient-boosted trees or regularized regression models, when such methods are specified or when they are used to approximate reported multivariable associations. For each analysis, the computer system 110 may store parameter estimates, such as regression coefficients, hazard ratios, odds ratios, or other summary measures, together with standard errors, confidence intervals, or other measures of uncertainty computed from the synthetic dataset. When the stochastic simulation engine 125 generates a plurality of whole-trial candidate datasets across iterations, the computer system 110 may execute the analyses of block 340 on each of the candidate datasets and may, in some embodiments, accumulate sets of parameter estimates across the plurality of candidate datasets to form empirical distributions of synthetic analysis results.

In some embodiments, executing statistical analyses 340 may also include organizing the synthetic analysis results into structures that facilitate subsequent comparison to benchmarks and optional meta-analytic assessments. The computer system 110 may aggregate the statistics computed at block 340 into a collection indexed by benchmark identifier, endpoint, arm comparison, and subgroup definition, so that for each benchmark the corresponding synthetic values can be retrieved efficiently at block 345. When analyses are applied across multiple synthetic datasets for the same trial, the computer system 110 may compute descriptive statistics over the collection of synthetic results for a given benchmark, such as means, medians, variances, or selected quantiles, and may store these synthetic distributions for later use in evaluating deviation from reported statistics, assessing coverage of benchmark values, or performing downstream meta-analyses across multiple trials.

In some embodiments, the method 300 may include determining one or more deviation scores and a bias metric 345. The computer system 110 may receive, as inputs to block 345, the statistical values computed from the whole-trial candidate dataset at block 340 and the corresponding reported statistical values in the set of benchmarks determined at block 330. For each benchmark, the computer system 110 may compute a deviation score that quantifies the difference between the synthetic value and the reported value. The deviation score for a given benchmark may be based on an absolute difference, a relative difference, a squared difference, a difference on a transformed scale (for example, log-hazard ratios or log-odds), or other distance measures. In some embodiments, the computer system 110 may normalize deviation scores by an estimate of variability, such as a reported standard error or confidence interval width, so that deviations for benchmarks with different scales can be compared on a common basis.

In some embodiments, determining deviation scores and the bias metric 345 may include aggregating deviation scores across multiple benchmarks using weights and penalty terms associated with constraint satisfaction. For example, the computer system 110 may assign higher weights to deviation scores for benchmarks corresponding to primary endpoints or key secondary endpoints, and lower weights to deviation scores for exploratory or secondary analyses. The computer system 110 may then combine the weighted deviation scores into an aggregate quantity, such as a weighted sum, a maximum deviation across benchmarks, or another monotonic function that increases as synthetic statistics diverge from reported values. In some embodiments, the computer system 110 may modify one or more deviation scores, or the aggregate quantity, by adding penalties when one or more constraints from the set of constraints determined at block 330 are not satisfied, such as when numerical tolerances are exceeded, clinical logical rules are violated, or subgroup definitions are not respected in the synthetic dataset. The resulting aggregate quantity may be recorded as a bias metric associated with the whole-trial candidate dataset.

In some embodiments, determining deviation scores and the bias metric 345 may also include constructing diagnostic information that is used for later analysis and for guiding subsequent iterations of the stochastic simulation. The computer system 110 may store, for each benchmark, the synthetic value computed at block 340, the corresponding reported value, and the deviation score, along with identifiers that specify the endpoint, arm comparison, subgroup definition, and analysis method. When a plurality of whole-trial candidate datasets is generated across iterations, the computer system 110 may track the bias metric over iterations and may compute empirical distributions of deviation scores and bias metrics across accepted or proposed datasets. These empirical distributions may be used to estimate bias, variability, and coverage properties of the synthetic datasets relative to the benchmarks, and may be consulted when configuring tolerance levels, weights, or penalty parameters. In some embodiments, the bias metric determined at block 345 is provided as an input to block 350, where it influences the determination of a probability value that controls whether the whole-trial candidate dataset is retained without change, modified, replaced, or transitioned to another candidate dataset within the stochastic simulation.

In some embodiments, the method 300 may include determining a probability value 350. The computer system 110 may receive, as inputs to block 350, the bias metric associated with the whole-trial candidate dataset determined at block 345 together with indications of whether the set of constraints determined at block 330 is satisfied for that dataset. The computer system 110 may then compute a probability value that is a function of the bias metric and of constraint satisfaction status, where the probability decreases as the bias metric increases or as more constraints are violated, and increases as the bias metric decreases and a larger fraction of constraints are satisfied. In some embodiments, the probability value may be computed using a monotonic transformation of the bias metric, such as an exponential or logistic function, possibly scaled by one or more temperature or tuning parameters that control the sensitivity of the probability to changes in the bias metric. In other embodiments, the probability value may be determined piecewise by comparing the bias metric to one or more thresholds and assigning different probability ranges according to which interval the bias metric falls into.

In some embodiments, determining the probability value 350 may include interpreting the probability value as part of a Markov chain or Markov-like stochastic process over synthetic datasets. The stochastic simulation engine 125 may maintain a current state corresponding to a previously accepted whole-trial synthetic dataset (for example, a state t) and may treat the whole-trial candidate dataset generated at block 335 as a proposed next state (for example, a state t+1). The probability value determined at block 350 may control the selection of at least one of several possible actions with respect to the proposed dataset, including retaining the current dataset without change, replacing the current dataset with the proposed dataset, modifying the proposed dataset through targeted adjustments, or transitioning to another candidate dataset generated by a further proposal step. For example, in some embodiments the computer system 110 may generate a random variate from a uniform distribution on the unit interval and compare the random variate to the probability value; if the random variate is less than the probability value, a particular action (such as accepting the proposed dataset as the new state or applying a specific adjustment operation) may be taken, while if the random variate exceeds the probability value, an alternative action (such as rejecting the proposed dataset and retaining the current state) may be taken. In other embodiments, the probability value may be used to define a discrete distribution over action types, and the computer system 110 may sample from that distribution to select which action to apply to the whole-trial candidate dataset.

In some embodiments, determining the probability value 350 and applying it to control actions on the whole-trial candidate dataset may be configured to support non-differentiable objective functions and complex constraint structures. For example, when the objective function that aggregates deviations and constraint penalties is non-differentiable or defined through discrete constraint violations, techniques based on gradient descent may not be suitable. Instead, the probability value at block 350 may be defined using acceptance rules similar to those used in Metropolis-Hastings or simulated annealing schemes, where the probability of moving to a proposed dataset depends on how the bias metric for the proposed dataset compares to the bias metric for the current dataset and on whether key constraints are satisfied. In some embodiments, the simulation may operate for many iterations, for example more than ten thousand iterations or more than one million iterations, with the probability value at each iteration influencing whether the process explores new regions of the synthetic dataset space, refines existing candidate datasets through incremental adjustments, or retains previously accepted datasets. When a whole-trial candidate dataset achieves a bias metric and constraint satisfaction pattern that meets predefined criteria, the corresponding state may be treated as a selected synthetic dataset and may be stored at block 355.

In some embodiments, the method 300 may include storing a synthetic dataset 355. The computer system 110 may identify one or more whole-trial candidate datasets generated at block 335 and evaluated at blocks 340, 345, and 350 that satisfy predefined quality criteria, such as having a bias metric below a tolerance threshold and satisfying a required proportion of the constraints determined at block 330. For each such dataset, the computer system 110 may store the synthetic patient-level records in the data store 135 as a synthetic representation of the clinical study. The stored records may include, for each synthetic participant, values for clinical variables, a trial arm identifier, and event-time and censoring indicators, together with any surrogate outcomes, subgroup labels, or derived quantities used during analysis. In some embodiments, the computer system 110 may also store, in association with the synthetic dataset, the bias metric, deviation scores for individual benchmarks, constraint satisfaction indicators, and identifiers for the simulation run, iteration number, and random seeds used to generate the dataset, thereby supporting reproducibility, auditing, and comparison across multiple synthetic datasets for the same trial.

In some embodiments, storing the synthetic dataset 355 may include organizing the stored data and metadata to support downstream analyses and cross-trial workflows. The computer system 110 may store synthetic datasets in a format compatible with statistical software, machine learning pipelines, or virtual trial simulators, and may register each synthetic dataset with the corresponding trial representation assembled at block 325 so that trial-level context, including temporal parameters, trial design attributes, and benchmark definitions, is available when the synthetic dataset is later retrieved. When multiple synthetic datasets are stored for a given trial, the computer system 110 may tag each dataset with a rank or quality indicator based on its bias metric or diagnostic summaries and may maintain collections of synthetic datasets that can be accessed for meta-analysis across trials, model training using multiple replicated datasets, or simulation of alternative trial designs. In other embodiments, the computer system 110 may export stored synthetic datasets to external systems via the user device 180 or network-based interfaces or may archive selected synthetic datasets for long-term storage while retaining summary statistics and diagnostic information in the data store 135 for rapid reference.

In some embodiments, the method may further include performing unsupervised ensemble machine learning using synthetic datasets generated as described herein. The computer system 110 may begin by creating a number N of synthetic datasets corresponding to a number J of clinical trials of interest, where each synthetic dataset represents patient-level records reconstructed from publication-level statistical summaries and trial metadata. Once created, the computer system 110 may merge the synthetic datasets from multiple trials to form a distribution of N replicated merged datasets, each merged dataset harmonizing variable definitions, factor levels, and units across trials. Following the merging process, the computer system 110 may apply inclusion and exclusion criteria to each merged dataset to generate N analysis datasets, for example by including synthetic patients who satisfy specified demographic or biomarker-defined criteria and excluding synthetic patients whose records are flagged as inconsistent or incomplete according to preconfigured quality checks.

In some embodiments, the method may include defining trial variables for use in unsupervised machine learning training. The computer system 110 may select, for each analysis dataset, a set of patient-level variables to serve as input features for the unsupervised models, such as demographic characteristics, baseline biomarker measurements, comorbidity indicators, treatment arm assignments, and endpoint results or derived outcome summaries. The computer system 110 may further specify one or more unsupervised learning methods and collections of hyperparameters for model training and for estimating low-dimensional embeddings of the trial variables. These hyperparameters may control aspects of the training process, such as the learning rate, regularization strength, neighborhood size, number of components, or the dimensionality of the embedding space. In some embodiments, method choices and hyperparameters may be configured by a user via the user device 180 or may be selected automatically by the computer system 110 using model selection heuristics.

In some embodiments, the method may include training unsupervised machine learning models in parallel across the N analysis datasets. For each dataset, the computer system 110 may instantiate an unsupervised model, such as an autoencoder, a manifold learning algorithm, a clustering-oriented embedding method, or another representation-learning approach, and may tune the model's hyperparameters to produce embeddings that capture relationships and patterns among the selected trial variables. The embeddings for each fitted model may map patients or variables into a high-dimensional or low-dimensional vector space in which proximity reflects similarity in characteristics or outcomes. Once embeddings are generated for all N datasets, the computer system 110 may, in some embodiments, project the embeddings into a common coordinate system and compute a composite embedding representation, for example by averaging or otherwise aggregating corresponding embedding coordinates across the N replicated datasets. From the composite embedding, the computer system 110 may construct an undirected graphical model in which nodes represent variables or patients and edges represent estimated associations or similarities, with edge weights reflecting the strength or stability of the relationships across the ensemble of synthetic datasets.

In some embodiments, the method may include applying community detection algorithms to the graphical model to identify clusters or communities of patients or variables that share similar characteristics. The computer system 110 may assign synthetic patients to local clusters based on graph structure, embedding distances, or both, and may profile the resulting clusters by examining the distributions of fitted variables and endpoints within each cluster. For example, clusters may reveal subtypes of patients who exhibit similar treatment responses, prognostic profiles, or risk factor combinations. The computer system 110 may validate the robustness of the identified clusters by evaluating their replicability across the N analysis datasets, for example by re-fitting models on different synthetic datasets and scoring the consistency of subtype prevalence and cluster characteristics. In some embodiments, cluster profiles and associated networks are ranked according to metrics such as cluster stability, concordance across datasets, complexity, and interpretability, and may be presented to users through graphical or tabular interfaces for further review and hypothesis generation. In other embodiments, the computer system 110 may iteratively adjust model choices, hyperparameters, or embedding procedures based on validation results to improve the quality and reproducibility of the discovered patterns.

In some embodiments, the method may further include performing supervised ensemble machine learning using synthetic datasets that correspond to a number N of replicated datasets for a number J of clinical trials of interest. The computer system 110 may create the N synthetic datasets using the generation methods described for the method 300 and may, for each trial, store patient-level synthetic records in the data store 135. The computer system 110 may then merge synthetic datasets from multiple trials to create a distribution of N replicated merged datasets, harmonizing trial-specific variables, factor levels, and measurement units to support cross-trial analysis. Once merged, the computer system 110 may apply inclusion and exclusion criteria to define N analysis datasets, for example by including synthetic patients belonging to specified trial arms or clinical subgroups and excluding synthetic records that fail to satisfy predefined quality thresholds or consistency checks. The N analysis datasets may be prepared for supervised training by defining, for each dataset, the outcomes to be predicted and the input variables to be used as features.

In some embodiments, the method may include specifying supervised learning parameters and model configurations for the ensemble of analysis datasets. The computer system 110 may define primary and secondary outcomes for each analysis dataset, such as time-to-event outcomes, binary response outcomes, or continuous endpoints, and may select independent variables and combinations of independent variables to serve as candidate feature sets for supervised training. The computer system 110 may further define k-fold indicators for cross-validation, partitioning each analysis dataset into k subsets for training and validation, and may select one or more supervised machine learning methods, such as regularized regression models, tree-based models, or neural network models. For each chosen method, the computer system 110 may define a collection of hyperparameters, such as learning rates, regularization strengths, tree depths, or network widths, and may identify one or more synthetic patient profiles to be used as reference or test profiles when inspecting model predictions.

In some embodiments, the method may include performing variable selection and model fitting across the N analysis datasets in parallel. For each analysis dataset, the computer system 110 may evaluate multiple candidate sets of input variables by fitting supervised models under a k-fold cross-validation scheme and computing cross-validation error or loss metrics for each candidate variable set. The computer system 110 may then rank variable combinations according to their cross-validation performance, identifying combinations that are most predictive of the primary and secondary outcomes. Using one or more of the top-ranked variable combinations, the computer system 110 may train supervised models in parallel for each of the N analysis datasets, applying the specified machine learning methods and hyperparameters. Each model may be fitted using k-fold cross-validation, where the dataset is repeatedly partitioned into training and validation folds, so that predictive performance can be assessed across multiple partitions.

In some embodiments, the method may include evaluating model performance, filtering the ensemble of models, and generating predictions using the selected models. For each fitted model, the computer system 110 may compare k-fold predicted outcomes to observed outcomes in the corresponding synthetic analysis dataset and may compute a concordance score, accuracy statistic, or other measure that quantifies alignment between predicted and observed values. The computer system 110 may then apply a filtering process to retain only those models whose concordance scores exceed a predefined threshold or satisfy other stability criteria, thereby reducing the model space to a subset of high-performing models. For this subset, the computer system 110 may generate predictions for specified patient profiles or for all synthetic records, and may, in some embodiments, select models for prediction in a probabilistic manner, with selection probabilities proportional to concordance scores or related performance metrics. The computer system 110 may further evaluate the concordance of predictions by comparing predicted outcomes to observed outcomes for patient profiles across all N synthetic datasets and, when available, across external validation cohorts, and may store summary statistics describing model performance and prediction robustness in the data store 135.

In some embodiments, the method may include iteratively adjusting supervised model configurations based on validation results to improve accuracy, stability, and reliability. The computer system 110 may modify the set of supervised methods under consideration, refine the hyperparameter spaces for individual methods, or update the variable selection process to reflect information gained from prior training runs, such as patterns of variable importance or systematic misprediction for particular subgroups. The computer system 110 may repeat variable selection, model fitting, and evaluation steps for one or more iterations, using updated configurations, until predefined performance criteria are met or a resource budget is exhausted. The resulting ensemble of supervised models, and their associated predictions, may then be used in downstream workflows, such as virtual trial simulation, risk stratification, or decision support analyses that operate on synthetic patient-level data.

In some embodiments, the method may further include performing semi-supervised ensemble machine learning using synthetic datasets that correspond to a number N of replicated datasets for a number J of clinical trials of interest. The computer system 110 may create the N synthetic datasets using the generation methods described for the method 300 and may merge synthetic datasets from multiple trials to create a distribution of N replicated merged datasets, with the merging process harmonizing trial-specific variables, factor levels, and measurement units as described for multi-trial merging. The computer system 110 may apply inclusion and exclusion criteria to each merged dataset to generate N analysis datasets, for example by including synthetic patients belonging to particular trial arms or patient subgroups and excluding synthetic records that do not satisfy predefined quality standards or consistency checks. Once the analysis datasets are prepared, the computer system 110 may define primary and secondary outcomes for supervised training, select independent variables and combinations of variables to serve as candidate feature sets, and associate these definitions with each analysis dataset.

In some embodiments, the method may include specifying embedding estimation procedures and collections of hyperparameters for semi-supervised model training. The computer system 110 may define one or more embedding methods and associated hyperparameters, such as regularization parameters, optimization algorithms, neighborhood sizes, and embedding dimensionalities, and may further identify patient profiles for validation and review. The computer system 110 may perform variable selection on each of the N analysis datasets, evaluating multiple candidate variable combinations under a supervised loss function, for example using k-fold cross-validation error as a selection criterion. The computer system 110 may rank candidate variable combinations according to their predictive performance for the specified outcomes, thereby prioritizing feature sets that are most informative for supervised prediction while still being suitable for subsequent embedding and clustering operations in the semi-supervised workflow.

In some embodiments, the method may include training semi-supervised models in parallel using the N analysis datasets and the selected variable combinations. For each dataset, the computer system 110 may apply a k-fold cross-validation scheme in which the data is partitioned into k subsets, the semi-supervised model is trained on k−1 subsets, and performance is evaluated on the remaining subset, incorporating both labeled outcome information and unsupervised representation learning. The computer system 110 may compare k-fold predicted outcomes to observed outcomes within each synthetic analysis dataset and compute concordance scores or related performance measures for the fitted models. The models may then be ranked according to concordance, and the computer system 110 may retain only those models whose performance exceeds a predefined threshold, thereby defining a filtered set of high-performing semi-supervised models. For the top-ranked models, the computer system 110 may project learned embeddings for patients or variables into a shared embedding space and compute a weighted average of projected embeddings across the filtered models, with weights that may depend on concordance or other performance metrics, to obtain a consolidated embedding representation.

In some embodiments, the method may include constructing a graphical representation and identifying subtypes using the consolidated embedding representation produced by the semi-supervised ensemble. The computer system 110 may construct an undirected graphical model in which nodes represent patients or variables and edges represent similarities or associations estimated from the averaged embeddings, with edge weights reflecting the strength or stability of relationships across the filtered models. The computer system 110 may apply community detection algorithms to this graphical model to assign patients to local clusters or subtypes based on shared characteristics and may profile the clusters by examining distributions of baseline covariates, treatment assignments, and endpoint results within each cluster. The computer system 110 may optionally generate predictions for primary and secondary outcomes using subtype assignments and local smoothing over edge weights and may validate the resulting subtype profiles using other synthetic datasets to assess the replicability of subtype prevalence and outcome patterns. The profiles and associated predictions may be ranked based on metrics such as outcome concordance, cluster stability, interpretability, and validation score, and may be presented through tabular or graphical interfaces or interactive exploration tools for review by automated systems or human analysts.

In some embodiments, the method may include adjusting semi-supervised model configurations based on validation results to improve the quality of subtype discovery and prediction. The computer system 110 may modify the set of semi-supervised methods under consideration, refine collections of hyperparameters for embedding estimation and supervised components, or update variable selection procedures to emphasize features that contribute to more stable and interpretable clusters. The computer system 110 may repeat one or more of the semi-supervised training, embedding, clustering, and validation steps using updated configurations until one or more performance, stability, or resource-usage criteria are satisfied. The resulting semi-supervised models, graphical representations, and cluster profiles may then be used in downstream workflows such as virtual trial simulation, risk stratification, or the design of targeted clinical studies based on synthetic patient-level data.

In some embodiments, the method may further include simulating a virtual clinical trial using one or more synthetic datasets and prediction models trained as described herein. The computer system 110 may obtain from the data store 135, one or more synthetic datasets corresponding to historical clinical trials that are relevant to a proposed virtual trial, for example based on indication, line of therapy, mechanism of action, or patient population. The computer system 110 may also obtain trial design parameters for the virtual trial, such as the number of virtual arms, arm definitions, randomization ratios, eligibility criteria, targeted sample sizes, numbers and locations of sites, site activation schedules, enrollment windows, follow-up durations, analysis time points, and success criteria defined in terms of one or more endpoint statistics. In some embodiments, the trial design parameters may be specified by a user through the user device 180, while in other embodiments they may be obtained from a design template or optimization routine.

In some embodiments, simulating a virtual clinical trial may include generating a population of virtual trial participants and assigning attributes to those participants based on the synthetic datasets and trial design parameters. The computer system 110 may construct virtual patient records that include non-stochastic attributes that remain fixed across replications (for example, trial-level parameters or scenario-level covariate distributions) and stochastic attributes that are sampled in each replication from one or more synthetic datasets. Stochastic attributes may include baseline covariates, biomarker values, comorbidity indicators, and latent risk factors, which may be sampled so that aggregate distributions in the virtual trial population are consistent with distributions observed in the synthetic datasets. The computer system 110 may assign each virtual patient to a trial arm according to the randomization ratios, assign the patient to a site, and generate a site activation date and patient-specific enrollment date according to site-level and trial-level enrollment schedules. Calendar-based follow-up windows and analysis cutoffs may be computed based on enrollment dates, site activation dates, and trial design parameters.

In some embodiments, simulating the virtual trial may include predicting outcomes for virtual patients using supervised or semi-supervised models trained on synthetic datasets. The computer system 110 may apply one or more prediction models to virtual patient records to obtain predicted primary and secondary outcomes, such as time-to-event endpoints, binary response indicators, or continuous outcome measures. Predictions may be generated in a manner that respects the temporal structure of the trial, for example by mapping predicted event times and censoring times to the trial calendar and truncating or censoring events outside interim or final analysis windows. For each replication of the virtual trial, the computer system 110 may compute endpoint statistics specified by the trial design, such as hazard ratios, response rate differences, or mean differences across arms, and may apply pre-specified analysis rules to determine whether the virtual trial would be declared successful under the success criteria (for example, a hazard ratio below a threshold with a confidence interval not crossing one, or a p-value below a specified significance level).

In some embodiments, the virtual trial simulation may be repeated for a plurality of replications, for example hundreds, thousands, or more replications, to obtain empirical distributions of endpoint statistics under the proposed design. For each replication, the computer system 110 may record whether the success criteria are met, along with the corresponding endpoint statistics and optionally intermediate measures such as enrollment patterns, event counts, and information fractions at interim analyses. The computer system 110 may then compute, from the collection of replications, empirical distributions for one or more endpoint statistics and may estimate a probability of success for the proposed trial design, for example by computing the proportion of replications in which the success criteria are satisfied. In some embodiments, the computer system 110 may present summary statistics, confidence intervals, or graphical representations of the predicted endpoint distributions and probability-of-success estimates through the user device 180 and may compare alternative virtual trial designs by repeating the simulation under different design parameter sets.

In some embodiments, the computer system 110 may maintain an integrated evidence database that aggregates synthetic datasets and associated metadata across multiple clinical studies. The integrated evidence database may include, for each trial, one or more top-ranked synthetic datasets selected according to bias metrics, diagnostic summaries, or other quality measures, as well as the corresponding trial representations, benchmark sets, and constraint sets. The integrated evidence database may organize trials by indication, disease stage, line of therapy, mechanism of action, geographic region, trial phase, and other attributes, and may store links to external references such as publications and registry entries. In some embodiments, the integrated evidence database may store additional cross-trial annotations, such as mappings between variable names, harmonized definitions of covariates and endpoints, and relationships between comparable treatment regimens across different studies.

In some embodiments, the integrated evidence database may be used to support cross-trial strategic intelligence and evaluation of future trial designs. The computer system 110 may execute queries over the integrated evidence database to summarize distributions of outcomes across trials, endpoints, patient subgroups, or treatment classes, and may compute comparative statistics that characterize performance of different therapies across diverse trial settings. The integrated evidence database may serve as a source of synthetic patient-level information and trial-level context for training machine learning models that generalize across multiple studies, and for configuring virtual trial simulations that draw from evidence accumulated across prior trials. In some embodiments, the computer system 110 may use the integrated evidence database to evaluate and prioritize candidate trial designs by estimating their probability of success under different assumptions, to explore potential effects of design changes such as modifications to eligibility criteria or sample sizes, and to support portfolio-level decisions regarding which indications, populations, or combinations of therapies to pursue.

In some embodiments, the computer system 110 may be implemented using a high-performance or distributed computing environment that allows the methods described herein to be executed at scale. For example, the computer system 110 may include tens, hundreds, or more processing cores, and may adjust the number of candidate datasets generated, the depth of the stochastic search over synthetic datasets, the number of virtual trial replications, or the number of machine learning models trained in parallel based on the available computational resources. In some embodiments, different components of the workflow, such as per-arm proposal steps, whole-trial evaluation, cross-trial merging, and ensemble model training, may be executed concurrently across multiple CPU cores, graphics processing units, tensor processing units, or other accelerator devices in a heterogeneous architecture. The computer system 110 may allocate tasks and manage parallel execution so that simulation, analysis, and virtual trial workflows complete within desired time windows even when operating on large numbers of synthetic datasets or complex trial designs.

In some embodiments, the stochastic simulation is executed concurrently across more than one hundred processing cores of the computer system 110, with different cores proposing and evaluating whole-trial candidate datasets in parallel. In some embodiments, the computer system 110 periodically identifies one or more whole-trial candidate datasets, or subsets of synthetic patient records, that exhibit lower bias metrics or better constraint satisfaction on a first set of processing cores and uses those candidate datasets or subsets as seeds to initialize or re-initialize simulations on other processing cores. In this manner, iterations on some cores may be re-seeded with better-scoring candidate simulated patient records generated on other cores while still proceeding independently to explore additional regions of the synthetic dataset space.

Some approaches to analyzing clinical trials based on publicly available information focus primarily on extracting or digitizing limited portions of reported results, such as approximating time-to-event outcomes from published survival plots or fitting parametric curves to summary endpoints, and then using those approximations for narrow secondary analyses. Such approaches often treat variables independently in ways that do not preserve multivariate relationships and yield reconstructed data that is not readily extensible beyond the specific endpoint depicted. In contrast, in some embodiments, a computing system may generate and iteratively refine candidate patient-level records across multiple variables using a set of constraints derived from a publication (for example, summary statistics, subgroup breakdowns, correlations, censoring patterns, trial design metadata, and reported analyses), may evaluate candidate datasets using one or more bias or fit metrics comparing reproduced analyses to reported outcomes, and may retain, update, or replace candidate datasets based on these metrics to obtain reconstructed datasets that satisfy a broader set of constraints. In some implementations, the resulting reconstructed datasets may be used not only to reproduce reported analyses, but also to support additional downstream workflows, such as harmonization across multiple studies, ensemble learning to identify patient subtypes, and repeated simulation of prospective trial designs, which may be less practical to implement using approaches limited to a single curve, endpoint, or univariate reconstruction.

Some embodiments may generate a reconstructed patient-level dataset that includes a plurality of patient records, each patient record, in some cases, being represented as a data structure having a defined set of fields that includes (i) an identifier (which may be synthetic), (ii) a treatment-arm indicator, (iii) one or more baseline covariates, and (iv) one or more outcome fields, where the outcome fields may include at least one time-to-event value and an associated censoring indicator and, in some implementations, one or more additional endpoints (such as longitudinal measurements, laboratory values, adverse-event indicators, response assessments, or other variables described in a publication). In some implementations, the system may generate candidate patient records such that, when aggregated, the reconstructed patient-level dataset satisfies a set of constraints derived from the publication across multiple reported variables, including constraints on marginal distributions (such as means, variances, proportions, or quantiles), cross-variable relationships (such as correlations, conditional distributions, subgroup effects, or stratified summaries), and analysis outputs (such as hazard ratios, response rates, Kaplan-Meier-derived quantities, or other reported statistics), and may evaluate and iteratively update the candidate patient records based on deviations between analyses performed on the reconstructed patient-level dataset and the reported analyses, thereby producing patient records that are usable for multivariable downstream analyses rather than being limited to an endpoint-specific reconstruction.

Some embodiments leverage the clinical data analysis platform, unified data format, and other techniques described in the following patent applications, the contents of which are hereby incorporated by reference:
a. U.S. Prov. Pat. App. 63/550,743|078525-0577457: SEARCHABLE AND NAVIGABLE ELECTRONIC SYSTEM FOR FINDING RESULTS FROM MANY ANALYSES APPLIED TO DATA;
b. U.S. Prov. Pat. App. 63/614,517|078525-0577453: REPRESENTING CLINICAL TRIAL DATA IN AN INTERACTIVE ANALYSIS PLATFORM;
c. U.S. patent application Ser. No. 18/990,773|078525-0581464: REPRESENTING CLINICAL TRIAL DATA IN AN INTERACTIVE ANALYSIS PLATFORM;
d. U.S. Prov. Pat. App. 63/614,516|078525-0577455: DATA INTEGRITY FOR CLINICAL STUDIES;
e. U.S. Prov. Pat. App. 63/567,392|078525-0579120: ENHANCING RETRIEVAL AUGMENTED GENERATION ACCURACY;
f. U.S. patent application Ser. No. 19/048,638|078525-0582553: ADAPTIVE CLINICAL TRIAL DATA ANALYSIS USING AI-GUIDED VISUALIZATION SELECTION; and
g. U.S. patent application Ser. No. 19/306,920|078525-0585445: ARTIFICIAL INTELLIGENCE APPLICATIONS TO ALERT A DATA SAFETY MONITORY BOARD TO ADVERSE EVENTS IN CLINICAL-TRIAL DATA.
As described above, some embodiments use one or more artificial intelligence (AI) techniques to generate synthetic patient records using published clinical trial results, without having access to the underlying individual patient records from the trial. The system may generate candidate synthetic patient records as structured data objects having defined fields, and may iteratively refine the candidate records by executing an objective or bias metric that quantifies deviation between analyses performed on the candidate dataset and analyses reported in the publication. In some cases, the iterative refinement may include sampling-based or Markov-chain-like transitions between candidate datasets, in which a probability value derived from the bias metric and constraint satisfaction is used to retain, modify, or replace candidate records, and in which proposed updates adjust distributions and cross-variable relationships to better satisfy the publication-derived constraints. In some implementations, the resulting synthetic patient-level datasets may be used as inputs to downstream machine-learning workflows, including ensemble clustering or graphical modeling to identify patient subtypes, supervised or semi-supervised outcome prediction trained on replicated reconstructions, and simulation of prospective trial designs by repeatedly sampling cohorts from reconstructed distributions and computing empirical success probabilities for specified endpoints.

In some embodiments, the system may implement a probabilistic machine-learning technique in which candidate patient-level datasets are treated as states in a stochastic generative process and are updated using Markov-chain transitions. In some implementations, the system may be configured in accordance with Markov chain Monte Carlo (MCMC) or related sampling-based inference methods used in artificial intelligence to sample from, or search over, high-dimensional distributions that are difficult to characterize in closed form. For example, the system may define an objective function or energy function based on (i) constraint satisfaction derived from published clinical trial results and (ii) one or more bias metrics quantifying deviation between analyses computed on a candidate dataset and analyses reported in a publication, and may generate proposals that modify one or more synthetic patient records or fields thereof. The system may then compute an acceptance probability for a proposed transition as a function of the objective function and may accept, reject, or otherwise select between candidate datasets based on the acceptance probability, thereby performing sampling-based probabilistic inference to generate synthetic patient records whose aggregated properties match the published constraints. In some cases, this sampling-based approach may be used to model dependencies among variables across patient records and to produce a distribution over feasible patient-level datasets consistent with the publication, which may be useful for downstream machine-learning tasks such as clustering, prediction, and simulation using generated patient records.

In some embodiments, the simulated patient records may be used as inputs to one or more downstream analyses that were not performed, or not reported, in the publication from which the constraints were derived. For example, in some cases the simulated patient records may be used to test hypotheses not addressed in the publication, such as evaluating an interaction between a baseline covariate and treatment response, estimating an effect within a subpopulation defined by a composite eligibility criterion, comparing alternative endpoint definitions, or assessing whether a response metric is associated with a downstream time-to-event outcome. In some implementations, the system or a downstream user may execute one or more statistical analyses on the simulated patient records, such as subgroup comparisons, Cox models, parametric survival models, logistic regression, longitudinal mixed-effects models, mediation analyses, or dose-response analyses, and may store or output resulting estimates, confidence intervals, p-values, or other measures of uncertainty derived from the simulated patient records.

Some embodiments may use emulated data in adaptions of machine learning and virtual trial simulation informed by emulated datasets. In some embodiments, the simulated patient records may be used to predict whether a future or ongoing clinical trial will be successful. In some implementations, a user may specify a proposed trial design including one or more candidate treatment arms, an enrollment schedule, eligibility criteria, endpoint definitions, follow-up duration, censoring assumptions, missing-data assumptions, interim analysis timing, or a statistical power target, and the system may simulate one or more virtual trials by sampling or constructing synthetic cohorts using the simulated patient records as a generative basis. In some cases, the system may repeatedly simulate trial outcomes and compute an empirical probability of meeting a success criterion, such as a hazard ratio exceeding a threshold, a p-value meeting a significance level, a response rate exceeding a target, or a safety event rate remaining below a limit. In some implementations, the system may support “what-if” exploration by varying one or more trial parameters (e.g., sample size, allocation ratio, enrichment strategy, endpoint choice, or duration) and generating corresponding forecasts of success probability, time-to-readout, expected effect size, or sensitivity to dropout.

In some embodiments, the simulated patient records may be used to form ensembles that aggregate information across multiple clinical trials, including trials of different therapies, indications, doses, regimens, or study populations. In some implementations, the system may generate simulated patient records for a plurality of publications and harmonize the records into a common schema by mapping variables, units, categorical levels, endpoint definitions, and follow-up windows, and may then create an ensemble dataset comprising multiple reconstructed cohorts. In some cases, the ensemble dataset may be used to estimate treatment efficacy or comparative effectiveness based on aggregate evidence, including by computing an overall effect estimate across trials, by performing anchored or unanchored indirect comparisons, by estimating treatment effects within harmonized subgroups, or by applying meta-analytic weighting based on sample size, variance, trial quality indicators, or measured heterogeneity. In some implementations, the system may quantify uncertainty by generating multiple simulated datasets per publication and propagating dataset-to-dataset variability into confidence intervals, posterior intervals, prediction intervals, or robustness scores for the aggregate efficacy conclusions.

In some embodiments, the simulated patient records may be used to train or evaluate machine-learning models. For example, in some cases the simulated patient records may be used to train predictive models of clinical endpoints (such as survival, progression, response, or adverse events), to identify patient subtypes that respond differently to treatment, or to learn representations of patient profiles that capture correlations among baseline covariates and outcomes. In some implementations, the system may use the simulated patient records to evaluate candidate biomarkers or composite risk scores by estimating their association with outcomes, by assessing calibration and discrimination metrics, or by simulating enrichment strategies that preferentially enroll predicted responders. In some cases, the simulated patient records may be used for external validation of a model trained on other sources, by comparing model predictions to endpoint distributions observed in the simulated patient records, including within subgroups or across trial arms.

In some embodiments, the simulated patient records may be used for operational, scientific, or regulatory-facing workflows. For example, in some cases a downstream user may use the simulated patient records to generate synthetic control arms when a randomized control is unavailable, impractical, or ethically constrained, and may compare outcomes from a prospective single-arm study to outcomes predicted from the simulated patient records while controlling for baseline covariates. In some implementations, the simulated patient records may be used to stress-test statistical analysis plans by executing alternative estimands, missing-data handling approaches, censoring assumptions, or multiplicity adjustments and measuring sensitivity of conclusions. In some cases, the simulated patient records may be used to perform quality checks on reported results by attempting to reproduce reported statistics under the publication-derived constraints, flagging inconsistencies when the system cannot construct datasets that jointly satisfy the reported constraints within tolerances, or identifying which reported values contribute most to reconstruction error. In some implementations, the simulated patient records may be used to compute health-economic outcomes, such as quality-adjusted life-year estimates or cost-effectiveness ratios, by attaching economic parameters to simulated clinical trajectories and propagating uncertainty via repeated simulation.

FIG. 13 is a diagram that illustrates an exemplary computing device 1000 in accordance with embodiments of the present technique, one or more of which may be networked to form a computing system in which the present techniques are implemented in some embodiments. A single computing device is shown, but some embodiments of a computer system may include multiple computing devices that communicate over a network, for instance in the course of collectively executing various parts of a distributed application. Various portions of systems and methods described herein, may include or be executed on one or more computer systems similar to computing system 1000. Further, processes and modules described herein may be executed by one or more processing systems similar to that of computing system 1000.

Computing system 1000 may include one or more processors (e.g., processors 1010a-1010n) coupled to system memory 1020, an input/output I/O device interface 1030, and a network interface 1040 via an input/output (I/O) interface 1050. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 1000. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 1020). Computing system 1000 may be a uni-processor system including one processor (e.g., processor 1010a), or a multi-processor system including any number of suitable processors (e.g., 1010a-1010n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 1000 may include a plurality of computing devices (e.g., distributed computer systems) to implement various processing functions.

I/O device interface 1030 may provide an interface for connection of one or more I/O devices 1060 to computer system 1000. I/O devices may include devices that receive input (e.g., from a user) or output information (e.g., to a user). I/O devices 1060 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 1060 may be connected to computer system 1000 through a wired or wireless connection. I/O devices 1060 may be connected to computer system 1000 from a remote location. I/O devices 1060 located on remote computer system, for example, may be connected to computer system 1000 via a network and network interface 1040.

Network interface 1040 may include a network adapter that provides for connection of computer system 1000 to a network. Network interface may 1040 may facilitate data exchange between computer system 1000 and other devices connected to the network. Network interface 1040 may support wired or wireless communication. The network may include an electronic communication network, such as the Internet, a local area network (LAN), a wide area network (WAN), a cellular communications network, or the like.

System memory 1020 may be configured to store program instructions 1100 or data 1110. Program instructions 1100 may be executable by a processor (e.g., one or more of processors 1010a-1010n) to implement one or more embodiments of the present techniques. Instructions 1100 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 1020 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 1020 may include a non-transitory computer readable storage medium that may have program instructions stored thereon that are executable by a computer processor (e.g., one or more of processors 1010a-1010n) to cause the subject matter and the functional operations described herein. A memory (e.g., system memory 1020) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 1050 may be configured to coordinate I/O traffic between processors 1010a-1010n, system memory 1020, network interface 1040, I/O devices 1060, and/or other peripheral devices. I/O interface 1050 may perform protocol, timing, or other data transformations to convert data signals from one component (e.g., system memory 1020) into a format suitable for use by another component (e.g., processors 1010a-1010n). I/O interface 1050 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 1000 or multiple computer systems 1000 configured to host different portions or instances of embodiments. Multiple computer systems 1000 may provide for parallel or sequential processing/execution of one or more portions of the techniques described herein.

Those skilled in the art will appreciate that computer system 1000 is merely illustrative and is not intended to limit the scope of the techniques described herein. Computer system 1000 may include any combination of devices or software that may perform or otherwise provide for the performance of the techniques described herein. For example, computer system 1000 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 1000 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 1000 may be transmitted to computer system 1000 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to costs constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B can include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and can be implemented in the form of data that causes functionality to be invoked, e.g., in the form of arguments of a function or API call. To the extent bespoke noun phrases (and other coined terms) are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

The present techniques will be better understood with reference to the following enumerated embodiments:
1. A method comprising: obtaining a publication reporting statistics resulting from a clinical trial; generating, without having access to real patient records in the clinical trial, simulated patient records consistent with the statistics in the publication by determining constraints on the simulated patient records from the publication and then, iteratively, until a stopping condition is detected: generate a new set of candidate simulated patient records by adjusting an initial set or current set of candidate simulated patient records; score the new set of candidate simulated patient records based on satisfaction of the constraints; and select a subset of the new set of candidate simulated patient records based on the scores as the current set of candidate simulated patient records; and storing at least some of the current set of candidate simulated patient records as the simulated patient records consistent with the statistics in the publication.
2. The method of embodiment 1, wherein adjusting comprises making a plurality of random or pseudorandom changes of less than a threshold amount to parameters in the initial set or current set of candidate simulated patient records.
3. The method of embodiment 2, comprising: selecting parameters to adjust based on an amount of mismatch relative to the constraints attributable to the parameters.
4. The method of embodiment 2, comprising: concurrently performing the iterations on more than 100 computing cores, re-seeding iterations on some of the cores with better scoring candidate simulated patients from other cores.
5. The method of embodiment 2, wherein the generated simulated patient records are generated with a sampling based optimization and not with a gradient-based optimization.
6. The method of embodiment 1, wherein a number of parameters in the generated simulated patient records is more than five and the number of constraints is more than five.
7. The method of embodiment 1, wherein computing the scores comprises executing Cox models, subgroup comparisons, and response calculations on the new set of candidate simulated patient records.
8. The method of embodiment 1, comprising: obtaining a first subset of the statistics in the publication from text in a table of the publication; and obtaining a second subset of the statistics in the publication from a curve in a graph of the publication.
9. The method of embodiment 1, wherein generating comprises: generating synthetic patient records in which univariate marginal probability distributions match corresponding values reported in the publication for each of a plurality of treatment arms in the clinical trial within a threshold tolerance.
10. The method of embodiment 1, comprising: using the generated simulated patient records to evaluate a hypothesis not addressed in the publication.
11. A tangible, non-transitory, machine-readable method storing instructions that when executed by a data processing apparatus cause the data processing apparatus to perform operations comprising: obtaining a publication reporting statistics resulting from a clinical trial, the publication comprising the following: a plurality of trial arms; a label for each trial arm; an indication of whether each trial arm was assigned as a treatment or control arm; a sample size for each trial arm; at least one clinical variable; at least one value correlating to reported distributions for the clinical variables, the reported distributions including one or more of the following: a mean, a standard deviation, a median, a range, or a proportion; and time-to-event information comprising Kaplan-Meier curve data and corresponding at-risk information; mapping time-to-event information to discrete time intervals and determining calibrated event-time and censoring indicators based on the mapped time-to-event information; determining, for each trial arm, a per-arm data structure, each data structure comprising reported distributions for clinical variables, categorical levels of clinical variables, and outcomes for that arm; assembling each of the per-arm data structures into a trial representation, the representation configured to store temporal parameters and to associate clinical variables with corresponding endpoints; determining a set of benchmarks comprising data describing the clinical trial, the data comprising one or more reported statistical values and indicating one or more statistical analysis methods used to produce the reported statistical values; determining a set of constraints based on the set of benchmarks and the obtained publication, the set of constraints bounding ranges of synthetic values for the clinical variables in accordance with the set of benchmarks and clinical logical rules of the clinical trial; initializing a stochastic simulation with a randomized seed and an initial candidate dataset; proposing, for each trial arm, a candidate set of synthetic patient records using candidate values selected using randomized sampling subject to the constraint set; combining the candidate set of synthetic patient records from each trial arm to form a whole-trial candidate dataset; executing, on the whole-trial candidate dataset, the one or more statistical analysis methods specified by the set of benchmarks and computing one or more statistical values from the whole-trial candidate dataset; determining a deviation score by comparing the one or more statistical values computed from the whole-trial candidate to corresponding reported statistical values in the set of benchmarks; determining a bias metric associated with the whole-trial candidate dataset based on the deviation score and whether the set of constraints is satisfied; determining, from the bias metric and a degree to which the set of constraints is satisfied, a probability value that is used to determine whether the whole-trial candidate dataset is retained without change, modified through adjustment of selected values, replaced with a newly proposed dataset, or transitioned to another candidate dataset within the stochastic simulation; and storing the whole-trial candidate dataset.
12. The method of embodiment 11, wherein the obtained publication further comprises: data identifying patient demographic characteristics comprising one or more of age, sex, race, or baseline clinical measures; data indicating hazard ratios for one or more clinical endpoints together with corresponding confidence intervals; data indicating response rates for one or more trial arms; and data indicating results for one or more reported clinical subgroups.
13. The method of embodiment 11, wherein the publication describing the clinical trial is obtained from one or more clinical trial publications or regulatory summaries, including at least one of peer-reviewed journal articles, conference abstracts, poster presentations, clinical trial reports, or regulatory documents such as FDA review memoranda or basis-of-approval summaries.
14. The method of embodiment 11, wherein the proposing, for each arm, a candidate set of synthetic patient values comprises: selecting variable values to satisfy reported per-arm univariate distributions; constructing multivariable patient profiles including demographics, clinical covariates, and treatment assignment consistent with endpoint definitions; and deriving patient-level event-time and censoring indicators from the mapped time-to-event information based on extracted Kaplan-Meier curve data, the corresponding at-risk information, and the discrete time intervals.
15. The method of embodiment 11, wherein determining a bias metric associated with the whole-trial candidate dataset is performed using a non-differentiable process that evaluates the deviation score together with satisfaction of the set of constraints without relying on gradient-based optimization.
16. The method of embodiment 11, wherein the set of benchmarks comprises data describing the clinical trial, the data including at least one of: data indicating hazard ratios with corresponding confidence intervals for one or more clinical endpoints; data indicating event rates or response rates for one or more trial arms; data indicating survival probabilities or median time-to-event values at specified time points derived from the time-to-event information; and data indicating summary statistical values for the clinical variables comprising one or more of a mean, a median, a standard deviation, a range, or a proportion.
17. The method of embodiment 11, wherein the operations are performed without accessing data comprising individual records for participants in the clinical trial, the individual records including data in which each record corresponds to a single participant and specifies one or more identifiers for the participant, a treatment assignment for the participant, or clinical measurements recorded for the participant.
18. The method of embodiment 11, wherein the set of constraints comprises clinical logical rules specifying permitted temporal and clinical relationships among events for the clinical trial, the clinical logical rules including at least one of the following: a rule that prevents a clinical event for a given participant from being assigned a time earlier than a start time associated with the participant; a rule that prevents a progression event or a response event for a given participant from being assigned a time later than a death event for the participant; a rule that requires any censoring time for a given participant to be on or before a last observed event time for the participant and within a follow-up period for the clinical trial; and a rule that requires all records for a given participant to remain associated with a single trial arm and a consistent treatment or control assignment.
19. The method of embodiment 11, further comprising: determining that the whole-trial candidate dataset fails to satisfy at least one of the set of constraints or is determined, based on the bias metric and the probability value; and performing one or more incrementation operations, the incrementation operations comprising: determining, for each combination of a clinical variable and an outcome associated with the clinical trial, a deviation value based on a difference between a statistical value computed from the whole-trial candidate dataset and a corresponding reported statistical value in the set of benchmarks; ranking the combinations of clinical variables and outcomes according to the deviation values to form an ordered list; selecting, from the ordered list, one or more of the clinical variables and associated patient records as targets for adjustment; adjusting the selected patient records by replacing values of the selected clinical variables with replacement values obtained using randomized sampling from per-arm distributions for the clinical variables subject to the set of constraints and consistent with the time-to-event information; and re-assembling the adjusted patient records for each trial arm together with unadjusted patient records to form an updated whole-trial candidate dataset for subsequent evaluation.
20. The method of embodiment 11, further comprising: determining, based on the probability value, that the whole-trial candidate dataset requires further modification; ranking variable- outcome combinations according to a magnitude of deviation from the benchmark statistical values; selecting one or more clinical variables and associated patient records for targeted adjustment using probabilistic re-sampling subject to the set of constraints; and re-assembling the adjusted patient records for each trial arm into an updated whole-trial candidate dataset for subsequent evaluation.
21. The method of embodiment 11, wherein determining the deviation score comprises applying one or more distance measurements to compare the one or more statistical values computed from the whole-trial candidate dataset to corresponding reported statistical values in the set of benchmarks, the distance measurements comprising at least one of: an absolute difference between a computed statistical value and a corresponding reported statistical value, a relative difference between a computed statistical value and a corresponding reported statistical value, a squared difference between a computed statistical value and a corresponding reported statistical value, and a difference between logarithms of a computed statistical value and a corresponding reported statistical value.
22. The method of embodiment 11, wherein determining the bias metric associated with the whole-trial candidate dataset comprises combining the deviation score with information indicating whether the set of constraints is satisfied such that contributions to the bias metric increase when one or more of the set of constraints is not satisfied or when deviations from the reported statistical values in the set of benchmarks increase, including at least one of: increasing the bias metric as a magnitude of deviation for a clinical variable and outcome combination increases, and increasing the bias metric as a number of unsatisfied constraints increases.
23. The method of embodiment 11, wherein mapping the time-to-event information to discrete time intervals comprises: defining a time grid for the clinical trial including a sequence of time points determined based on at least one of changes in the Kaplan-Meier curve data and times at which the corresponding at-risk information is reported; and assigning event-time and censoring indicators to time intervals defined by adjacent time points in the time grid.
24. The method of embodiment 11, wherein the operations further comprise: repeating the operations of proposing, combining, and evaluating the whole-trial candidate dataset one or more times, wherein an additional whole-trial candidate dataset is created each time the operations are repeated; determining a bias metric for each of the additional whole-trial candidate datasets; ranking the whole-trial candidate datasets according to the bias metrics; and selecting one or more of the whole-trial candidate datasets having bias metrics within a lowest range of the ranked bias metrics for storage or analysis.
25. The method of embodiment 11, wherein the operations are performed in a distributed computing environment comprising a plurality of cloud computing systems, and wherein the stochastic simulation is executed in parallel across the plurality of cloud computing systems to generate whole-trial candidate datasets using separate processing resources that each perform proposing, combining, and evaluating operations for one or more of the whole-trial candidate datasets.
26. The method of embodiment 11, wherein the operations further comprise: receiving user input specifying one or more of: which reported statistical values in the data describing the clinical trial are included in the set of benchmarks; a tolerance value for one or more of the reported statistical values indicating an acceptable range of deviation between the reported statistical values and corresponding statistical values computed from the whole-trial candidate dataset; and a relative weighting for one or more of the reported statistical values indicating an influence of deviation for the reported statistical values on the bias metric.
27. The method of embodiment 11, wherein the operations further comprise: training a machine-learning model using the whole-trial candidate dataset as training data, the training comprising: supplying clinical variables, treatment assignments, and event-time and outcome indicators from the whole-trial candidate dataset as inputs to the machine-learning model; and adjusting parameters of the machine-learning model to reduce prediction error for one or more clinical endpoints.
28. The method of embodiment 11, wherein the operations further comprise simulating a virtual clinical trial, the simulating comprising: selecting, from the whole-trial candidate dataset, synthetic patient records that satisfy one or more eligibility criteria for the virtual clinical trial; assigning the selected synthetic patient records to one or more virtual treatment arms according to a specified allocation scheme; constructing a virtual enrollment schedule for the selected synthetic patient records; and computing simulated trial outcomes and summary statistics for the virtual treatment arms based on treatment assignments, event-time indicators, and outcome indicators in the selected synthetic patient records.
29. The method of embodiment 11, wherein determining a set of constraints comprises steps for comparing the data describing the clinical trial and the set of benchmarks to identify numerical bounds and clinical logical rules that bound admissible synthetic values for the clinical variables.
30. The medium of embodiment 11, wherein determining the set of benchmarks and the set of constraints further comprises: creating, for each trial arm, a per-arm data structure comprising reported distributions for clinical variables, categorical levels of the clinical variables, and associated outcomes; assembling the per-arm data structures into a trial representation configured to store temporal parameters and associate the clinical variables with corresponding endpoints; translating information from the publication into the trial representation to define and store target benchmark values along with one or more statistical analysis methods, wherein the stored analysis methods are configured for repeated implementation on each candidate synthetic dataset to compute comparable statistics; determining a deviation score and bias metric by repeatedly applying the stored analysis methods to the candidate synthetic datasets and comparing the computed statistics to the stored target benchmark values; and bounding admissible synthetic values for the clinical variables based on the target benchmark values and clinical logical rules determined from the publication.
31. The medium of embodiment 11, wherein the operations further comprise using the whole-trial candidate dataset to design a future clinical trial by simulating modifications to parameters including at least one of eligibility criteria, sample size, enrollment rates, or follow-up durations, and computing probabilities of success or failure based on empirical distributions determined from the dataset.
32. The medium of embodiment 11, wherein the operations further comprise establishing benchmarks for clinical trial performance using the whole-trial candidate dataset, the benchmarks including normalized time-to-event distributions adjusted for varying accrual rates or follow-up durations, and evaluating deviations in outcomes across multiple emulated trials.
33. The medium of embodiment 11, wherein the operations further comprise merging the whole-trial candidate dataset with emulated datasets from other clinical trials to perform a meta-analysis, including harmonizing variables, imputing missing values, and analyzing multivariate relationships not directly reported in the publication or publications of the other clinical trials.
34. The medium of embodiment 11, wherein the operations further comprise analyzing the whole-trial candidate dataset to characterize patient heterogeneity, including computing distributions of clinical variables, prognostic factors, and enrollment trends across subgroups or trials.
35. The medium of embodiment 11, wherein the operations further comprise evaluating adverse event risks and efficacy trade-offs using the whole-trial candidate dataset, including computing hazard ratios or risk differences across therapeutic classes, specific drugs, or clinical indications.
36. The medium of embodiment 11, wherein the operations further comprise integrating the whole-trial candidate dataset with observed patient-level data from an accessible clinical trial, including aligning variables and computing comparative distributions for outcomes or subgroups.
37. The medium of embodiment 11, wherein the operations further comprise monitoring an ongoing clinical trial by comparing interim data to benchmarks derived from the whole-trial candidate dataset and determining underperformance or outperformance to support ethical decision-making.
38. The medium of embodiment 11, wherein the operations further comprise applying biostatistical analyses, machine learning models, or simulation procedures to the whole-trial candidate dataset to interrogate unreported relationships, predict outcomes, or evaluate anomalous conclusions across studies.
39. The medium of embodiment 11, wherein the operations further comprise evaluating investment opportunities in drug development using the whole-trial candidate dataset, including assessing unmet needs or promising sectors based on aggregated efficacy and safety distributions.
40. The medium of embodiment 11, wherein the operations further comprise using the whole-trial candidate dataset in at least one of: reliability simulations for trial conclusions, deviation analyses in outcome distributions, or any analytical procedure requiring patient-level data emulation without direct access to original records.
41. The medium of embodiment 11, wherein the operations further comprise comparing the whole-trial candidate dataset to a set of candidate trial designs and selecting a design based on metrics including statistical power, type I error rate, or expected accrual time.
42. The medium of embodiment 11, wherein the operations further comprise computing bias metrics for differential patient enrollment using the whole-trial candidate dataset and generating benchmarks for therapeutic mechanism of action based on transition rates between disease states at standardized follow-up intervals.
43. The medium of embodiment 11, wherein the operations further comprise normalizing outcome distributions across multiple emulated datasets for standardized follow-up durations and computing summary statistics for heterogeneity in efficacy or safety endpoints.
44. The medium of embodiment 11, wherein the operations further comprise comparing distributions of patient characteristics and outcomes in the whole-trial candidate dataset to those from other emulated or observed datasets to identify differences in populations or prognostic impacts.
45. The medium of embodiment 11, wherein the operations further comprise interrogating differential performance in efficacy and toxicity by applying statistical models to the whole-trial candidate dataset and identifying class effects versus drug-specific effects.
46. The medium of embodiment 11, wherein the operations further comprise generating counterfactual predictions using the whole-trial candidate dataset by simulating modifications to inclusion/exclusion criteria or treatment assignments and identifying anomalous data points in an ongoing trial.
47. The medium of embodiment 11, wherein the operations further comprise defining statistical operating characteristics for an ongoing trial using the whole-trial candidate dataset, including probabilities of ethical violations based on historical evidence.
48. The medium of embodiment 11, wherein the operations further comprise using the whole-trial candidate dataset to improve treatment protocols by analyzing transition rates between disease states and generating recommendations based on subgroup-specific responses.
49. The medium of embodiment 11, wherein the operations further comprise extracting strategic intelligence from the whole-trial candidate dataset to guide decisions on therapeutic development, including comparisons of performance across disease indications or drug classes.
50. A tangible, non-transitory, machine-readable medium storing instructions that when executed by a data processing apparatus cause the data processing apparatus to perform operations comprising: the operations of any one of embodiments 1-49
51. A system, comprising: one or more processors; and memory storing instructions that when executed by the processors cause the processors to effectuate operations comprising: the operations of any one of embodiments 1-49.

What is claimed is:

1. A tangible, non-transitory, machine-readable medium storing instructions that when executed by a computer system cause the computer system to effectuate operations comprising:
obtaining, with a computer system, a publication reporting statistics resulting from a clinical trial;
generating, with the computer system, without having access to real patient records in the clinical trial, simulated patient records consistent with the statistics in the publication by determining constraints on the simulated patient records from the publication and then, iteratively, until a stopping condition is detected:
generating a new set of candidate simulated patient records by adjusting an initial set or current set of candidate simulated patient records,
scoring the new set of candidate simulated patient records based on satisfaction of the constraints, and
selecting a subset of the new set of candidate simulated patient records based on the scores as the current set of candidate simulated patient records; and
storing, with the computer system, at least some of the current set of candidate simulated patient records as the simulated patient records consistent with the statistics in the publication.

2. The medium of claim 1, wherein adjusting comprises making a plurality of random or pseudorandom changes of less than a threshold amount to parameters in the initial set or current set of candidate simulated patient records.

3. The medium of claim 2, the operations comprising:
selecting parameters to adjust based on an amount of mismatch relative to the constraints attributable to the parameters.

4. The medium of claim 2, the operations comprising:
concurrently performing the iterations on more than 100 computing cores, re-seeding iterations on some of the cores with better scoring candidate simulated patient records from other cores.

5. The medium of claim 2, wherein the generated simulated patient records are generated with a sampling based optimization and not with a gradient-based optimization.

6. The medium of claim 1, wherein a number of parameters in the generated simulated patient records is more than five and the number of constraints is more than five.

7. The medium of claim 1, wherein computing the scores comprises executing Cox models, subgroup comparisons, and response calculations on the new set of candidate simulated patient records.

8. The medium of claim 1, the operations comprising:
obtaining a first subset of the statistics in the publication from text in a table of the publication; and
obtaining a second subset of the statistics in the publication from a curve in a graph of the publication.

9. The medium of claim 1, the operations comprising:
using the generated simulated patient records to evaluate a hypothesis not addressed in the publication, wherein generating comprises:
generating synthetic patient records in which univariate marginal probability distributions match corresponding values reported in the publication for each of a plurality of treatment arms in the clinical trial within a threshold tolerance.

10. The medium of claim 1, wherein generating the simulated patient records comprises generating a simulated clinical trial dataset; obtaining the publication comprises obtaining a publicly available document that reports the statistics and that excludes any subject-level dataset of the clinical trial, wherein the statistics in the publication include (i) at least one baseline characteristic statistic for participants in the clinical trial, (ii) at least one reported treatment-effect statistic, and (iii) at least one time-to-event statistic, wherein the simulated patient records each comprise a respective data structure including a synthetic patient identifier field, a treatment-arm field, at least one baseline covariate field, and at least one outcome field that includes a time-to-event value and a censoring indicator, wherein determining the constraints from the publication comprises determining a plurality of constraints including at least one marginal-distribution constraint, at least one stratified or subgroup constraint, and at least one constraint on a relationship between two different fields of the simulated patient records, wherein scoring the new set of candidate simulated patient records comprises (a) computing, from the new set of candidate simulated patient records, at least one analysis output corresponding to at least one of the reported treatment-effect statistic or the time-to-event statistic and (b) computing, as at least a portion of the scores, a deviation metric between the analysis output and a counterpart statistic reported in the publication, wherein selecting the subset of the new set of candidate simulated patient records comprises selecting, based on the scores, a best-scoring subset as the current set of candidate simulated patient records, and wherein the stopping condition is detected based on the deviation metric satisfying a threshold for the plurality of constraints.

11. A tangible, non-transitory, machine-readable medium storing instructions that when executed by a computer system cause the computer system to perform operations comprising:
obtaining, with a computer system, a publication reporting statistics resulting from a clinical trial, the publication comprising the following:
a plurality of trial arms;
a label for each trial arm;
an indication of whether each trial arm was assigned as a treatment or control arm;
a sample size for each trial arm;
at least one clinical variable;
at least one value correlating to reported distributions for the clinical variables, the reported distributions including one or more of the following: a mean, a standard deviation, a median, a range, or a proportion; and
time-to-event information comprising Kaplan-Meier curve data and corresponding at-risk information;
mapping, with the computer system, time-to-event information to discrete time intervals and determining calibrated event-time and censoring indicators based on the mapped time-to-event information;
determining, with the computer system, for each trial arm, a per-arm data structure, each data structure comprising reported distributions for clinical variables, categorical levels of clinical variables, and outcomes for that arm;
assembling, with the computer system, each of the per-arm data structures into a trial representation, the representation configured to store temporal parameters and to associate clinical variables with corresponding endpoints;
determining, with the computer system, a set of benchmarks comprising data describing the clinical trial, the data comprising one or more reported statistical values and indicating one or more statistical analysis methods used to produce the reported statistical values;
determining, with the computer system, a set of constraints based on the set of benchmarks and the obtained publication, the set of constraints bounding ranges of synthetic values for the clinical variables in accordance with the set of benchmarks and clinical logical rules of the clinical trial;
initializing, with the computer system, a stochastic simulation with a randomized seed and an initial candidate dataset;
proposing, with the computer system, for each trial arm, a candidate set of synthetic patient records using candidate values selected using randomized sampling subject to the set of constraints;
combining, with the computer system, the candidate set of synthetic patient records from each trial arm to form a whole-trial candidate dataset;
executing, with the computer system, on the whole-trial candidate dataset, the one or more statistical analysis methods specified by the set of benchmarks and computing one or more statistical values from the whole-trial candidate dataset;
determining, with the computer system, a deviation score by comparing the one or more statistical values computed from the whole-trial candidate dataset to corresponding reported statistical values in the set of benchmarks;
determining, with the computer system, a bias metric associated with the whole-trial candidate dataset based on the deviation score and whether the set of constraints is satisfied;
determining, with the computer system, from the bias metric and a degree to which the set of constraints is satisfied, a probability value that is used to determine whether the whole-trial candidate dataset is retained without change, modified through adjustment of selected values, replaced with a newly proposed dataset, or transitioned to another candidate dataset within the stochastic simulation; and
storing, with the computer system, the whole-trial candidate dataset.

12. The medium of claim 11, wherein the obtained publication further comprises:
data identifying patient demographic characteristics comprising one or more of age, sex, race, or baseline clinical measures;
data indicating hazard ratios for one or more clinical endpoints together with corresponding confidence intervals;
data indicating response rates for one or more trial arms; and
data indicating results for one or more reported clinical subgroups.

13. The medium of claim 11, wherein the publication describing the clinical trial is obtained from one or more clinical trial publications or regulatory summaries, including at least one of peer-reviewed journal articles, conference abstracts, poster presentations, clinical trial reports, or regulatory documents such as FDA review memoranda or basis-of-approval summaries.

14. The medium of claim 11, wherein the proposing, for each arm, a candidate set of synthetic patient records comprises:
selecting variable values to satisfy reported per-arm univariate distributions;
constructing multivariable patient profiles including demographics, clinical covariates, and treatment assignment consistent with endpoint definitions; and
deriving patient-level event-time and censoring indicators from the mapped time-to-event information based on extracted Kaplan-Meier curve data, the corresponding at-risk information, and the discrete time intervals.

15. The medium of claim 11, wherein determining a bias metric associated with the whole-trial candidate dataset is performed using a non-differentiable process that evaluates the deviation score together with satisfaction of the set of constraints without relying on gradient-based optimization.

16. The medium of claim 11, wherein the set of benchmarks comprises data describing the clinical trial, the data including at least one of:
data indicating hazard ratios with corresponding confidence intervals for one or more clinical endpoints;
data indicating event rates or response rates for one or more trial arms;
data indicating survival probabilities or median time-to-event values at specified time points derived from the time-to-event information; and
data indicating summary statistical values for the clinical variables comprising one or more of a mean, a median, a standard deviation, a range, or a proportion.

17. The medium of claim 11, wherein the operations are performed without accessing data comprising individual records for participants in the clinical trial, the individual records including data in which each record corresponds to a single participant and specifies one or more identifiers for the participant, a treatment assignment for the participant, or clinical measurements recorded for the participant.

18. The medium of claim 11, wherein the set of constraints comprises clinical logical rules specifying permitted temporal and clinical relationships among events for the clinical trial, the clinical logical rules including at least one of the following:
a rule that prevents a clinical event for a given participant from being assigned a time earlier than a start time associated with the participant;
a rule that prevents a progression event or a response event for a given participant from being assigned a time later than a death event for the participant;
a rule that requires any censoring time for a given participant to be on or before a last observed event time for the participant and within a follow-up period for the clinical trial; and
a rule that requires all records for a given participant to remain associated with a single trial arm and a consistent treatment or control assignment.

19. The medium of claim 11, the operations further comprising:
determining that the whole-trial candidate dataset fails to satisfy at least one of the set of constraints; and
performing one or more incrementation operations, the incrementation operations comprising:
determining, for each combination of a clinical variable and an outcome associated with the clinical trial, a deviation value based on a difference between a statistical value computed from the whole-trial candidate dataset and a corresponding reported statistical value in the set of benchmarks;
ranking the combinations of clinical variables and outcomes according to the deviation values to form an ordered list; selecting, from the ordered list, one or more of the clinical variables and associated patient records as targets for adjustment;
adjusting the selected patient records by replacing values of the selected clinical variables with replacement values obtained using randomized sampling from per-arm distributions for the clinical variables subject to the set of constraints and consistent with the time-to-event information; and
re-assembling the adjusted patient records for each trial arm together with unadjusted patient records to form an updated whole-trial candidate dataset for subsequent evaluation.

20. The medium of claim 11, the operations further comprising:
determining, based on the probability value, that the whole-trial candidate dataset requires further modification;
ranking variable-outcome combinations according to a magnitude of deviation from the benchmark statistical values;
selecting one or more clinical variables and associated patient records for targeted adjustment using probabilistic re-sampling subject to the set of constraints; and
re-assembling the adjusted patient records for each trial arm into an updated whole-trial candidate dataset for subsequent evaluation.

21. The medium of claim 11, wherein determining the deviation score comprises applying one or more distance measurements to compare the one or more statistical values computed from the whole-trial candidate dataset to corresponding reported statistical values in the set of benchmarks, the distance measurements comprising at least one of: an absolute difference between a computed statistical value and a corresponding reported statistical value, a relative difference between a computed statistical value and a corresponding reported statistical value, a squared difference between a computed statistical value and a corresponding reported statistical value, and a difference between logarithms of a computed statistical value and a corresponding reported statistical value.

22. The medium of claim 11, wherein determining the bias metric associated with the whole-trial candidate dataset comprises combining the deviation score with information indicating whether the set of constraints is satisfied such that contributions to the bias metric increase when one or more of the set of constraints is not satisfied or when deviations from the reported statistical values in the set of benchmarks increase, including at least one of: increasing the bias metric as a magnitude of deviation for a clinical variable and outcome combination increases, and increasing the bias metric as a number of unsatisfied constraints increases.

23. The medium of claim 11, wherein mapping the time-to-event information to discrete time intervals comprises:
defining a time grid for the clinical trial including a sequence of time points determined based on at least one of changes in the Kaplan-Meier curve data and times at which the corresponding at-risk information is reported; and
assigning event-time and censoring indicators to time intervals defined by adjacent time points in the time grid.

24. The medium of claim 11, wherein the operations further comprise:
repeating the operations of proposing, combining, and evaluating the whole-trial candidate dataset one or more times, wherein an additional whole-trial candidate dataset is created each time the operations are repeated;
determining a bias metric for each of the additional whole-trial candidate datasets;
ranking the whole-trial candidate datasets according to the bias metrics; and
selecting one or more of the whole-trial candidate datasets having bias metrics within a lowest range of the ranked bias metrics for storage or analysis.

25. The medium of claim 11, wherein the operations are performed in a distributed computing environment comprising a plurality of cloud computing systems, and wherein the stochastic simulation is executed in parallel across the plurality of cloud computing systems to generate whole-trial candidate datasets using separate processing resources that each perform proposing, combining, and evaluating operations for one or more of the whole-trial candidate datasets.

26. The medium of claim 11, wherein the operations further comprise:
receiving user input specifying one or more of:
which reported statistical values in the data describing the clinical trial are included in the set of benchmarks;
a tolerance value for one or more of the reported statistical values indicating an acceptable range of deviation between the reported statistical values and corresponding statistical values computed from the whole-trial candidate dataset; and
a relative weighting for one or more of the reported statistical values indicating an influence of deviation for the reported statistical values on the bias metric.

27. The medium of claim 11, wherein the operations further comprise:
training a machine-learning model using the whole-trial candidate dataset as training data, the training comprising:
supplying clinical variables, treatment assignments, and event-time and outcome indicators from the whole-trial candidate dataset as inputs to the machine-learning model; and
adjusting parameters of the machine-learning model to reduce prediction error for one or more clinical endpoints.

28. The medium of claim 11, wherein the operations further comprise simulating a virtual clinical trial, the simulating comprising:
selecting, from the whole-trial candidate dataset, synthetic patient records that satisfy one or more eligibility criteria for the virtual clinical trial;
assigning the selected synthetic patient records to one or more virtual treatment arms according to a specified allocation scheme;
constructing a virtual enrollment schedule for the selected synthetic patient records; and
computing simulated trial outcomes and summary statistics for the virtual treatment arms based on treatment assignments, event-time indicators, and outcome indicators in the selected synthetic patient records.

29. The medium of claim 11, wherein determining a set of constraints comprises steps for comparing the data describing the clinical trial and the set of benchmarks to identify numerical bounds and clinical logical rules that bound admissible synthetic values for the clinical variables.

30. The medium of claim 11, wherein determining the set of benchmarks and the set of constraints further comprises:
creating, for each trial arm, a per-arm data structure comprising reported distributions for clinical variables, categorical levels of the clinical variables, and associated outcomes;
assembling the per-arm data structures into a trial representation configured to store temporal parameters and associate the clinical variables with corresponding endpoints;
translating information from the publication into the trial representation to define and store target benchmark values along with one or more statistical analysis methods, wherein the stored analysis methods are configured for repeated implementation on each candidate synthetic dataset to compute comparable statistics;
determining a deviation score and bias metric by repeatedly applying the stored analysis methods to the candidate synthetic datasets and comparing the computed statistics to the stored target benchmark values; and
bounding admissible synthetic values for the clinical variables based on the target benchmark values and clinical logical rules determined from the publication.

31. The medium of claim 11, wherein the operations further comprise:
using the whole-trial candidate dataset to design a future clinical trial by simulating modifications to parameters including at least one of eligibility criteria, sample size, enrollment rates, or follow-up durations, and computing probabilities of success or failure based on empirical distributions determined from the dataset;
establishing benchmarks for clinical trial performance using the whole-trial candidate dataset, the benchmarks including normalized time-to-event distributions adjusted for varying accrual rates or follow-up durations, and evaluating deviations in outcomes across multiple emulated trials;
merging the whole-trial candidate dataset with emulated datasets from other clinical trials to perform a meta-analysis, including harmonizing variables, imputing missing values, and analyzing multivariate relationships not directly reported in the publication or publications of the other clinical trials;
analyzing the whole-trial candidate dataset to characterize patient heterogeneity, including computing distributions of clinical variables, prognostic factors, and enrollment trends across subgroups or trials;
evaluating adverse event risks and efficacy trade-offs using the whole-trial candidate dataset, including computing hazard ratios or risk differences across therapeutic classes, specific drugs, or clinical indications;
integrating the whole-trial candidate dataset with observed patient-level data from an accessible clinical trial, including aligning variables and computing comparative distributions for outcomes or subgroups;
monitoring an ongoing clinical trial by comparing interim data to benchmarks derived from the whole-trial candidate dataset and determining underperformance or outperformance to support ethical decision-making;
applying biostatistical analyses, machine learning models, or simulation procedures to the whole-trial candidate dataset to interrogate unreported relationships, predict outcomes, or evaluate anomalous conclusions across studies;
evaluating investment opportunities in drug development using the whole-trial candidate dataset, including assessing unmet needs or promising sectors based on aggregated efficacy and safety distributions;
using the whole-trial candidate dataset in at least one of: reliability simulations for trial conclusions, deviation analyses in outcome distributions, or any analytical procedure requiring patient-level data emulation without direct access to original records;
comparing the whole-trial candidate dataset to a set of candidate trial designs and selecting a design based on metrics including statistical power, type I error rate, or expected accrual time;
computing bias metrics for differential patient enrollment using the whole-trial candidate dataset and generating benchmarks for therapeutic mechanism of action based on transition rates between disease states at standardized follow-up intervals;
normalizing outcome distributions across multiple emulated datasets for standardized follow-up durations and computing summary statistics for heterogeneity in efficacy or safety endpoints;
comparing distributions of patient characteristics and outcomes in the whole-trial candidate dataset to those from other emulated or observed datasets to identify differences in populations or prognostic impacts;
interrogating differential performance in efficacy and toxicity by applying statistical models to the whole-trial candidate dataset and identifying class effects versus drug-specific effects;
generating counterfactual predictions using the whole-trial candidate dataset by simulating modifications to inclusion/exclusion criteria or treatment assignments and identifying anomalous data points in an ongoing trial;
defining statistical operating characteristics for an ongoing trial using the whole-trial candidate dataset, including probabilities of ethical violations based on historical evidence;
using the whole-trial candidate dataset to improve treatment protocols by analyzing transition rates between disease states and generating recommendations based on subgroup-specific responses; or
extracting strategic intelligence from the whole-trial candidate dataset to guide decisions on therapeutic development, including comparisons of performance across disease indications or drug classes.

32. A method comprising:
obtaining, with a computer system, a publication reporting statistics resulting from a clinical trial, the publication comprising the following:
a plurality of trial arms;
a label for each trial arm;
an indication of whether each trial arm was assigned as a treatment or control arm;
a sample size for each trial arm;
at least one clinical variable;
at least one value correlating to reported distributions for the clinical variables, the reported distributions including one or more of the following: a mean, a standard deviation, a median, a range, or a proportion; and
time-to-event information comprising Kaplan-Meier curve data and corresponding at-risk information;
mapping, with the computer system, time-to-event information to discrete time intervals and determining calibrated event-time and censoring indicators based on the mapped time-to-event information;
determining, with the computer system, for each trial arm, a per-arm data structure, each data structure comprising reported distributions for clinical variables, categorical levels of clinical variables, and outcomes for that arm;
assembling, with the computer system, each of the per-arm data structures into a trial representation, the representation configured to store temporal parameters and to associate clinical variables with corresponding endpoints;
determining, with the computer system, a set of benchmarks comprising data describing the clinical trial, the data comprising one or more reported statistical values and indicating one or more statistical analysis methods used to produce the reported statistical values;
determining, with the computer system, a set of constraints based on the set of benchmarks and the obtained publication, the set of constraints bounding ranges of synthetic values for the clinical variables in accordance with the set of benchmarks and clinical logical rules of the clinical trial;
initializing, with the computer system, a stochastic simulation with a randomized seed and an initial candidate dataset;
proposing, with the computer system, for each trial arm, a candidate set of synthetic patient records using candidate values selected using randomized sampling subject to the set of constraints;
combining, with the computer system, the candidate set of synthetic patient records from each trial arm to form a whole-trial candidate dataset;
executing, with the computer system, on the whole-trial candidate dataset, the one or more statistical analysis methods specified by the set of benchmarks and computing one or more statistical values from the whole-trial candidate dataset;
determining, with the computer system, a deviation score by comparing the one or more statistical values computed from the whole-trial candidate dataset to corresponding reported statistical values in the set of benchmarks;
determining, with the computer system, a bias metric associated with the whole-trial candidate dataset based on the deviation score and whether the set of constraints is satisfied;
determining, with the computer system, from the bias metric and a degree to which the set of constraints is satisfied, a probability value that is used to determine whether the whole-trial candidate dataset is retained without change, modified through adjustment of selected values, replaced with a newly proposed dataset, or transitioned to another candidate dataset within the stochastic simulation; and
storing, with the computer system, the whole-trial candidate dataset.
